(19) United States
(12) Patent Application Publication
(10) Pub. No.: US 2022/0260552 A1
(43) Pub. Date: Aug. 18, 2022

(54) Methods to determine the effect of an agent on mammalian embryonic development

(71) Applicant: Toxys B.V., Oegstgeest (NL)

(72) Inventors: Giel Hendriks, Rotterdam (NL); Inger Brandsma, Delfgauw (NL); Péter Imre Racz, Rijnsburg (NL)

(21) Appl. No.: 17/627,853

(22) PCT Filed: Jul. 17, 2020
(86) PCT No.: PCT/NL2020/050471

§ 371 Date: Jan. 18, 2022

(30) Foreign Application Priority Data
Jul. 19, 2019 (NL) 2023536

(51) Int. Cl. G01N 33/50; C12N 15/113

(52) U.S. Cl. CPC G01N 33/5023; C12N 15/113

(57) ABSTRACT

The invention relates to the fields of developmental toxicity. In particular, it relates to novel reporter cell types that may be used in in vitro methods to determine the effect of an agent on mammalian embryonic development.

### FIELD OF THE INVENTION

The invention relates to the field of developmental toxicity. In particular, it relates to novel reporter cell types that may be used in in vitro methods to determine the effect of an agent on mammalian embryonic development.

### BACKGROUND OF THE INVENTION

Embryonic development of organisms follows an extremely accurate pattern of differentiation processes, starting with early differentiation of embryonic stem cells into three primary germ cell lineages (ectoderm, mesoderm and endoderm). All the different tissues of the adult organism are derived from these three lineages. The differentiation process that is responsible for the transformation of the germ cells into the different cell types needed for these tissues, is a tightly timed and controlled process that is highly evolutionary conserved between species. Perturbation of differentiation processes by exposure to chemicals and xenobiotics may interfere with embryonic development and can result in severe birth defects, including physical malformations, abnormal behavior and cognitive problems.

Testing for disruption of embryonic development is a crucial part of toxicological risk assessment for novel compounds. Regulatory agencies demand rigorous testing of all novel compounds for potential developmental toxicity effects for humans, prior to releasing new products to the market. Currently, developmental and reproductive toxicity (DART) tests are heavily depending on in vivo studies (OECD TG 414, OECD TG 415, OECD TG 416, OECD TG 421, OECD TG 422).

As an alternative to these standard in vivo studies, several innovative in vitro studies have been developed. In vitro assays can be performed in a shorter timeframe than in vivo studies and help to significantly reduce the number of animals required for testing by removing agents causing developmental toxicity in vitro from the testing process in vivo. Furthermore, in vitro assays allow for high-throughput screening of multiple compounds during early screening processes to enable early prioritization, optimization and selection of compounds.

Various in vitro approaches have been developed to partly replace the in vivo reproductive toxicity test. An in vitro assay based on mouse cells is the embryonic stem cell test (EST). In the EST embryonic stem cells are differentiated towards functional beating cardiomyocytes while the cells are treated with the test compound during the complete differentiation process (Ellis-hutchings et al, 2010). Developmental toxicity is defined as a reduction of the number of beating cardiomyocytes after exposure to a test substance. The EST has a number of important limitations, including the lack of homogeneity of the differentiated population, the unpredictability and difficulties in quantification of the result. Furthermore, human specific teratogens, such as thalidomide, cannot be detected.

Another well-known approach is the Whole Embryo Culture (WEC), wherein complete rat embryos of approximately day 10 of gestation are cultured and exposed to potential toxins for up to 48 hours. Afterwards, cell viability, embryo function, growth and morphology are the endpoints deciding the toxic effects of the possibly teratogenic compounds. However, WEC has several disadvantages amongst them the technical requirements and the short culture duration which can cause misinterpretation of results, especially in classifying weak teratogens (Ellis-hutchings supra, Piersma et al 1993).

Another method to measure developmental toxicity is the ReProGlo stem cell-based Wnt reporter assay. This assay is based on the Wnt/β-catenin pathway, which plays a pivotal role in embryonic development. As the level of Wnt signalling can be measured by luciferase activity, a luciferase reporter is made to identify compounds affecting the Wnt pathway. Although the assay is accurate in rejecting false positives, it has a relatively high number of false negatives (Uibel and Schwarz, 2015). Thus, the Wnt reporter assay is not reliable enough to become the leading assay to test teratogenicity.

Thus, although in the last decades various in vitro assays have been developed, improving the field of developmental toxicity, most of these tests have only a low predictive value for human risk assessments and are only able to partially replace the existing in vivo tests. The demand for innovative in vitro assays which are easy-to-use and have a high predictive value for humans remains.

### SUMMARY OF THE INVENTION

In a first aspect, the invention provides for an in vitro method of determining an effect of an agent on mammalian embryonic development, the method comprising:

a) providing one or more types of reporter cells, wherein each type of reporter cell comprises a reporter sequence operatively linked to a different regulatory element of a gene;

b) contacting the one or more type of reporter cells with the agent;

c) comparing the expression of the reporter sequences in the one or more types of reporter cells contacted with the agent to a corresponding cell not contacted with the agent; and

d) determining that the agent has an effect on mammalian embryonic development if in step c) a difference in expression of the reporter sequences is detected between the reporter cells contacted with the agent and the corresponding cells not contacted with the agent for at least one type of reporter cell; and,

wherein the different regulatory elements for each type of reporter cell is selected from the group consisting of:
a regulatory element of the OCT4 gene comprising a polynucleotide sequence that has at least 60% sequence identity with at least one of SEQ ID NO: 1 and SEQ ID NO: 21;
a regulatory element of the BMP4 gene, comprising a polynucleotide sequence that has at least 60% sequence identity at least one of SEQ ID NO: 2 and SEQ ID NO: 22;
a regulatory element of the MYH6 gene, comprising a polynucleotide sequence that has at least 60% sequence identity with at least one of SEQ ID NO: 3 and SEQ ID NO:23;
a regulatory element of the PAX6 gene, comprising a polynucleotide sequence that has at least 60% sequence identity with at least one of SEQ ID NO: 4 and SEQ ID NO: 24;
a regulatory element of the FOXA2 gene, comprising a polynucleotide sequence that has at least 60% sequence identity with at least one of SEQ ID NO: 5 and SEQ ID NO: 25;
a regulatory element of the SOX17 gene, comprising a polynucleotide sequence that has at least 60% sequence identity with at least one of SEQ ID NO: 6 and SEQ ID NO: 26;
a regulatory element of the ALB gene, comprising a polynucleotide sequence that has at least 60% sequence identity with at least one of SEQ ID NO: 7 and SEQ ID NO: 27;
a regulatory element of the AFP gene, comprising a polynucleotide sequence that has at least 60% sequence identity with at least one of SEQ ID NO: 8 and SEQ ID NO: 28;
a regulatory element of the Ck18 gene comprising a polynucleotide sequence that has at least 60% sequence identity with at least one of SEQ ID NO: 9 and SEQ ID NO: 29;
a regulatory element of the Vegfr1 gene comprising a polynucleotide sequence that has at least 60% sequence identity with at least one of SEQ ID NO: 10 and SEQ ID NO: 30; and
a regulatory element of the SOX1 gene comprising a polynucleotide sequence that has at least 60% sequence identity with at least one of SEQ ID NO: 101 and SEQ ID NO: 103.

In a preferred embodiment, the method of the invention comprises contacting at least seven types of reporter cells with the agent in step b), wherein the at least seven reporter cells consist of a reporter cell comprising the regulatory element of OCT4 as herein described, a reporter cell comprising the regulatory element of BMP4 as herein described, a reporter cell comprising the regulatory element of MYH6 as herein described, a reporter cell comprising the regulatory element of FOXA2 as herein described, a reporter cell comprising the regulatory element of AFP as herein described, a reporter cell comprising the regulatory element of SOX1 as herein described, a reporter cell comprising the regulatory element of PAX6 as herein described,

In a second aspect, the invention provides for kit of parts comprising:
one or more types of reporter cell, wherein each type of reporter cell comprises a reporter sequence operatively linked to a regulatory element of a gene and wherein the one or more type of reporter cells each comprise a different regulatory element selected from:
a regulatory element of the OCT4 gene comprising a polynucleotide sequence that has at least 60% sequence identity with at least one of SEQ ID NO: 1 and SEQ ID NO: 21;
a regulatory element of the BMP4 gene, comprising a polynucleotide sequence that has at least 60% sequence identity at least one of SEQ ID NO: 2 and SEQ ID NO: 22;
a regulatory element of the MYH6 gene, comprising a polynucleotide sequence that has at least 60% sequence identity with at least one of SEQ ID NO: 3 and SEQ ID NO:23;
a regulatory element of the PAX6 gene, comprising a polynucleotide sequence that has at least 60% sequence identity with at least one of SEQ ID NO: 4 and SEQ ID NO: 24;
a regulatory element of the FOXA2 gene, comprising a polynucleotide sequence that has at least 60% sequence identity with at least one of SEQ ID NO: 5 and SEQ ID NO: 25;
a regulatory element of the SOX17 gene, comprising a polynucleotide sequence that has at least 60% sequence identity with at least one of SEQ ID NO: 6 and SEQ ID NO: 26;
a regulatory element of the ALB gene, comprising a polynucleotide sequence that has at least 60% sequence identity with at least one of SEQ ID NO: 7 and SEQ ID NO: 27;
a regulatory element of the AFP gene, comprising a polynucleotide sequence that has at least 60% sequence identity with at least one of SEQ ID NO: 8 and SEQ ID NO: 28;
a regulatory element of the Ck18 gene comprising a polynucleotide sequence that has at least 60% sequence identity with at least one of SEQ ID NO: 9 and SEQ ID NO: 29;
a regulatory element of the Vegfr1 gene comprising a polynucleotide sequence that has at least 60% sequence identity with at least one of SEQ ID NO: 10 and SEQ ID NO: 30; and
a regulatory element of the SOX1 gene comprising a polynucleotide sequence that has at least 60% sequence identity with at least one of SEQ ID NO: 101 and SEQ ID NO: 103.

In a preferred embodiment, the kit of parts of the invention comprises at least seven types of reporter cells consisting of a reporter cell comprising the regulatory element of OCT4 as herein described, a reporter cell comprising the regulatory element of BMP4 as herein described, a reporter cell comprising the regulatory element of MYH6 as herein described, a reporter cell comprising the regulatory element of FOXA2 as herein described, a reporter cell comprising the regulatory element of AFP as herein described, a reporter cell comprising the regulatory element of SOX1 as herein described, a reporter cell comprising the regulatory element of PAX6 as herein described.

In a third aspect, the invention provides for a combination of at least two transgenic non-human mammals, preferably rodents, comprising at least one cell comprising a reporter sequence operatively linked to a different regulatory element and wherein the regulatory element is selected from:
a regulatory element of the OCT4 gene comprising a polynucleotide sequence that has at least 60% sequence identity with at least one of SEQ ID NO: 1 and SEQ ID NO: 21;
a regulatory element of the BMP4 gene, comprising a polynucleotide sequence that has at least 60% sequence identity at least one of SEQ ID NO: 2 and SEQ ID NO: 22;
a regulatory element of the MYH6 gene, comprising a polynucleotide sequence that has at least 60% sequence identity with at least one of SEQ ID NO: 3 and SEQ ID NO: 23;
a regulatory element of the PAX6 gene, comprising a polynucleotide sequence that has at least 60% sequence identity with at least one of SEQ ID NO: 4 and SEQ ID NO: 24;
a regulatory element of the FOXA2 gene, comprising a polynucleotide sequence that has at least 60% sequence identity with at least one of SEQ ID NO: 5 and SEQ ID NO: 25;
a regulatory element of the SOX17 gene, comprising a polynucleotide sequence that has at least 60% sequence identity with at least one of SEQ ID NO: 6 and SEQ ID NO: 26;
a regulatory element of the ALB gene, comprising a polynucleotide sequence that has at least 60% sequence identity with at least one of SEQ ID NO: 7 and SEQ ID NO: 27;
a regulatory element of the AFP gene, comprising a polynucleotide sequence that has at least 60% sequence identity with at least one of SEQ ID NO: 8 and SEQ ID NO: 28;
a regulatory element of the Ck18 gene comprising a polynucleotide sequence that has at least 60% sequence identity with at least one of SEQ ID NO: 9 and SEQ ID NO: 29;
a regulatory element of the Vegfr1 gene comprising a polynucleotide sequence that has at least 60% sequence identity with at least one of SEQ ID NO: 10 and SEQ ID NO: 30; and
a regulatory element of the SOX1 gene comprising a polynucleotide sequence that has at least 60% sequence identity with at least one of SEQ ID NO: 101 and SEQ ID NO: 103.

In a preferred embodiment, invention relates for a combination of at least seven transgenic non-human mammals comprising at least one cell comprising a reporter sequence operatively linked to a different regulatory element, wherein the regulatory elements comprise the regulatory element of OCT4 as herein described, the regulatory element of BMP4 as herein described the regulatory element of MYH6 as herein described, the regulatory element of FOXA2 as herein described, the regulatory element of AFP as herein described the regulatory element of SOX1 as herein described, the regulatory element of PAX6 as herein described.

In a fourth aspect, the invention provides for, the use of one or more types of reporter cells for the determination of an effect of an agent on mammalian embryonic development, wherein each type of reporter cell comprises a reporter sequence operatively linked to a different regulatory element of a gene, and wherein the different regulatory elements for each type of reporter cell is selected from the group consisting of:
a regulatory element of the OCT4 gene comprising a polynucleotide sequence that has at least 60% sequence identity with at least one of SEQ ID NO: 1 and SEQ ID NO: 21;
a regulatory element of the BMP4 gene, comprising a polynucleotide sequence that has at least 60% sequence identity at least one of SEQ ID NO: 2 and SEQ ID NO: 22;
a regulatory element of the MYH6 gene, comprising a polynucleotide sequence that has at least 60% sequence identity with at least one of SEQ ID NO: 3 and SEQ ID NO: 23;
a regulatory element of the PAX6 gene, comprising a polynucleotide sequence that has at least 60% sequence identity with at least one of SEQ ID NO: 4 and SEQ ID NO: 24;
a regulatory element of the FOXA2 gene, comprising a polynucleotide sequence that has at least 60% sequence identity with at least one of SEQ ID NO: 5 and SEQ ID NO: 25;
a regulatory element of the SOX17 gene, comprising a polynucleotide sequence that has at least 60% sequence identity with at least one of SEQ ID NO: 6 and SEQ ID NO: 26;
a regulatory element of the ALB gene, comprising a polynucleotide sequence that has at least 60% sequence identity with at least one of SEQ ID NO: 7 and SEQ ID NO: 27;
a regulatory element of the AFP gene, comprising a polynucleotide sequence that has at least 60% sequence identity with at least one of SEQ ID NO: 8 and SEQ ID NO: 28;
a regulatory element of the Ck18 gene comprising a polynucleotide sequence that has at least 60% sequence identity with at least one of SEQ ID NO: 9 and SEQ ID NO: 29;
a regulatory element of the Vegfr1 gene comprising a polynucleotide sequence that has at least 60% sequence identity with at least one of SEQ ID NO: 10 and SEQ ID NO: 30; and
a regulatory element of the SOX1 gene comprising a polynucleotide sequence that has at least 60% sequence identity with at least one of SEQ ID NO: 101 and SEQ ID NO: 103.

In a preferred embodiment, the invention provides for a use as described herein, comprising at least seven types of reporter cells, wherein the at least seven reporter cells are a reporter cell comprising the regulatory element of OCT4 as herein described, a reporter cell comprising the regulatory element of BMP4 as herein described, a reporter cell comprising the regulatory element of MYH6 as herein described, a reporter cell comprising the regulatory element of FOXA2 as herein described, a reporter cell comprising the regulatory element of AFP as herein described, a reporter cell comprising the regulatory element of SOX1 as herein described, a reporter cell comprising the regulatory element of PAX6 as herein described.

In a fifth aspect, the invention provides for the reporter cells as defined herein. In one embodiment, the invention provides for a collection of reporter cells as defined herein.

### DETAILED DESCRIPTION OF THE INVENTION

The inventors have identified a panel of biomarker genes that are transcriptionally activated or deactivated during different phases of early stem cell differentiation and embryonic tissue development. Based on these identified genes, the inventors have generated a panel human induced pluripotent stem cells (hiPSC) and mouse embryonic stem cells (mES) reporter cell lines. These reporter cells include a reporter sequence operatively linked to a regulatory element of one of the selected genes thereby allowing the visualization and quantitative assessment of disturbance of the specific signaling pathways during different stages of embryonic differentiation. This system provides the possibility to detect various cell type-specific teratogenic properties in a differentiating multicellular culture thereby substantially improving the classification of substances for their hazard in early human embryonic development. Furthermore, because the reporter sequence is included in every cell of the cell line, the read-out of the assay can be quantified at the single cell level, resulting in increased sensitivity and specificity.

Accordingly, in a first aspect, the invention provides an in vitro method of determining an effect of an agent on mammalian embryonic development. The method comprising:
a) providing one or more types of reporter cells, wherein each type of reporter cell comprises a reporter sequence operatively linked to a different regulatory element of a gene;
b) contacting the one or more type of reporter cells with the agent;
c) comparing the expression of the reporter sequences in the one or more types of reporter cells contacted with the agent to a corresponding cell not contacted with the agent; and,
d) determining that the agent has an effect on mammalian embryonic development if in step c) a difference in expression of the reporter sequences is detected between the reporter cells contacted with the agent and the corresponding cells not contacted with the agent for at least one type of reporter cell; and,
wherein the different regulatory elements for each type of reporter cell is selected from the group consisting of:
   a regulatory element of the OCT4 gene comprising a polynucleotide sequence that has at least 60% sequence identity with SEQ ID NO: 1 or SEQ ID NO: 21;
   a regulatory element of the BMP4 gene, comprising a polynucleotide sequence that has at least 60% sequence identity with SEQ ID NO: 2 or SEQ ID NO: 22;
   a regulatory element of the MYH6 gene, comprising a polynucleotide sequence that has at least 60% sequence identity with SEQ ID NO: 3 or SEQ ID NO: 23;
   a regulatory element of the PAX6 gene, comprising a polynucleotide sequence that has at least 60% sequence identity with SEQ ID NO: 4 or SEQ ID NO: 24;
   a regulatory element of the FOXA2 gene, comprising a polynucleotide sequence that has at least 60% sequence identity with SEQ ID NO: 5 or SEQ ID NO: 25;
   a regulatory element of the SOX17 gene, comprising a polynucleotide sequence that has at least 60% sequence identity with SEQ ID NO: 6 or SEQ ID NO: 26;
   a regulatory element of the ALB gene, comprising a polynucleotide sequence that has at least 60% sequence identity with SEQ ID NO: 7 or SEQ ID NO: 27;
   a regulatory element of the AFP gene, comprising a polynucleotide sequence that has at least 60% sequence identity with SEQ ID NO: 8 or SEQ ID NO: 28;
   a regulatory element of the Ck18 gene comprising a polynucleotide sequence that has at least 60% sequence identity with SEQ ID NO: 9 or SEQ ID NO: 29;
   a regulatory element of the Vegfr1 gene comprising a polynucleotide sequence that has at least 60% sequence identity with SEQ ID NO: 10 or SEQ ID NO: 30; and
   a regulatory element of the SOX1 gene comprising a polynucleotide sequence that has at least 60% sequence identity with at least one of SEQ ID NO: 101 and SEQ ID NO: 103.

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

Methods of comparing the expression of the reporter sequences in the one or more types of reporter cells contacted with the agent to a corresponding cell not contacted with the agent (i.e. a control cell) are known to the person skilled in the art. Methods include but are not limited to fluorescence microscopy, high-content imaging, qPCR, RT-PCR, RNA sequencing, western blot, imaging flow cytometry and flow cytometry.

A difference in expression of the reporter sequences can be any kind of observed difference.

For example, the level of expression of the reporter sequence is an increase, a decrease, or no change in the level of expression of the reporter sequence as compared to the basal transcription level of the diagnostic nucleic acid or polypeptide. In one embodiment, the desired level of expression of reporter sequences is a decrease in the level of expression of the reporter sequence as compared to the basal transcription level of the reporter sequence.

In one embodiment, the reporter cell is a cell of a reporter cell line, a non-naturally occurring cell or a cultured cell.

As described above, the differentiation process from a fertilized oocyte into the primary embryonic germ layer cells (ectoderm, mesoderm and endoderm) and subsequently to the various embryonic tissues and organs, is a tightly timed and controlled process that is highly evolutionary conserved between species. The inventors have identified a number of genes that are highly representative for the differentiation processes at various stages of the embryonic development. Disturbance of the timely expression of these genes indicates disruption of proper embryonic development. The reporter cells lines as described herein can be deployed to determine disturbance of an abnormal pattern of expression of these genes that are linked to reporter sequence following exposure to an agent (as compared to a reporter cell that has not been exposed to the agent). Thus, by exposing one or more of the types of reporter cells as described herein to an agent, it is possible to categorize that agent as having a teratogenic effect. Accordingly, the present invention provides for a method of predicting teratogenicity of an agent.

OCT4 (octamer-binding transcription factor 4, POU5F1) is a transcription factor expressed in the pregastrulation embryo, early cleavage stage embryo, cells of the inner cell mass of the blastocyst, and in embryonic carcinoma (EC) cells. During normal development, OCT4 is down-regulated when cells are induced to differentiate in vitro and in the adult OCT4 is only found in germ cells. Disturbances in OCT4 expression can thus indicate that an agent thus has an effect on the early stages of embryonic development. During in vitro differentiation of hiPSC, OCT4 expression decreases overtime (FIG. 1A).

Preferably, the regulatory element of the OCT4 gene comprises or consists of a polynucleotide sequence that has at least 65, 70, 75, 80, 85, 90, 95, 96, 97, 98, 99 or 100% sequence identity with at least one of SEQ ID NO: 1 and SEQ ID NO: 21, of which SEQ ID NO: 1 is more preferred. More preferably, the regulatory element of the OCT4 gene comprises or consists of a polynucleotide sequence that has at least 65, 70, 75, 80, 85, 90, 95, 96, 97, 98, 99 or 100% sequence identity with at least one of SEQ ID NO: 11 and SEQ ID NO: 31 of which SEQ ID NO: 11 is more preferred.

BMP4 (Bone morphogenetic protein 4) is a transcription factor expressed during the middle stages of embryonic development and is essential during embryogenesis, most prominently for mesoderm formation and cardiac development. BMP4 has further been described to be involved in inducing cartilage and bone formation, tooth development, limb formation and fracture repair. In vitro during differentiation, BMP4 expression steadily increases and peaks around day 7, after which BMP4 expression slowly declines (see FIG. 1A). Disturbances in BMP4 expression can thus indicate that an agent thus has an effect on the middle stages of embryonic development.

Preferably, the regulatory element of the BMP4 gene comprises or consists of a polynucleotide sequence that has at least 65, 70, 75, 80, 85, 90, 95, 96, 97, 98, 99 or 100% sequence identity with at least one of SEQ ID NO: 2 and SEQ ID NO: 22, of which SEQ ID NO: 2 is more preferred. More preferably, the regulatory element of the BMP4 gene comprises or consists of a polynucleotide sequence that has at least 65, 70, 75, 80, 85, 90, 95, 96, 97, 98, 99 or 100% sequence identity with at least one of SEQ ID NO: 12 and SEQ ID NO: 32, of which SEQ ID NO: 12 is more preferred.

MYH6 (Myosin heavy chain, a isoform) is a motor protein that plays a role in muscle contraction and is expressed in mature cardiomyocytes. During embryonic differentiation, MYH6 expression increases over time, and peaks towards the end of the process (see FIG. 1A). Disturbances in MYH6 expression can thus indicate that an agent thus has an effect on the late stages of embryonic development.

Preferably, the regulatory element of the MYH6 gene comprises or consists of a polynucleotide sequence that has at least 65, 70, 75, 80, 85, 90, 95, 96, 97, 98, 99 or 100% sequence identity with at least one of SEQ ID NO: 3 and SEQ ID NO: 23, of which SEQ ID NO: 3 is more preferred. More preferably, the regulatory element of the MYH6 gene comprises or consists of a polynucleotide sequence that has at least 65, 70, 75, 80, 85, 90, 95, 96, 97, 98, 99 or 100% sequence identity with at least one of SEQ ID NO: 13 and SEQ ID NO: 33, of which SEQ ID NO: 13 is more preferred.

PAX6 (Paired box protein) also plays a role in embryological development. The PAX6 gene, found on chromosome 2, can be seen expressed in multiple early structures such as the spinal cord, hindbrain, forebrain and eyes. Mutations of the PAX6 gene in mammalian species can produce a drastic effect on the phenotype of the organism. PAX6 is also expressed in neural rosettes. During differentiation, PAX6 expression increases over time, and peaks towards the end of the neural rosette formation process (see FIG. 1C). Disturbances in PAX6 expression can thus indicate that an agent thus has an effect on the middle stages of embryonic development.

Preferably, the regulatory element of the PAX6 gene comprises or consists of a polynucleotide sequence that has at least 65, 70, 75, 80, 85, 90, 95, 96, 97, 98, 99 or 100% sequence identity with at least one of SEQ ID NO: 4 and SEQ ID NO: 24, of which SEQ ID NO: 4 is more preferred. More preferably, the regulatory element of the PAX6 gene comprises or consists of a polynucleotide sequence that has at least 65, 70, 75, 80, 85, 90, 95, 96, 97, 98, 99 or 100% sequence identity with at least one of SEQ ID NO: 14 and SEQ ID NO: 34, of which SEQ ID NO: 14 is more preferred.

SOX1 is a gene that encodes a transcription factor with a high mobility group DNA binding domain. SOX1 is one of the earliest transcription factors to be expressed in ectodermal cells committed to the neural fate: the onset of expression of SOX1 appears to coincide with the induction of neural ectoderm. During differentiation, SOX1 expression steadily increases and peaks around day 7, after which SOX1 expression slowly declines (see FIG. 1C). Disturbances in PAX6 expression can thus indicate that an agent thus has an effect on the middle stages of embryonic development.

Preferably, the regulatory element of the SOX1 gene comprises or consists of a polynucleotide sequence that has at least 65, 70, 75, 80, 85, 90, 95, 96, 97, 98, 99 or 100% sequence identity with at least one of SEQ ID NO: 101 and SEQ ID NO: 103, of which SEQ ID NO: 101 is more preferred. More preferably, the regulatory element of the SOX1 gene comprises or consists of a polynucleotide sequence that has at least 65, 70, 75, 80, 85, 90, 95, 96, 97, 98, 99 or 100% sequence identity with at least one of SEQ ID NO: 102 and SEQ ID NO: 104, of which SEQ ID NO: 102 is more preferred.

FOXA2 (forkhead box protein A2) is required during embryonic development for notochord formation and is involved in the development of multiple endoderm-derived organ systems such as the liver, pancreas and lungs. During embryonic differentiation, FOXA2 expression steadily increases and peaks around day 7, after which FOXA2 expression slowly declines (see FIG. 1B). Disturbances in FOXA2 expression can thus indicate that an agent thus has an effect on the intermediate stages of embryonic development.

Preferably, the regulatory element of the FOXA2 gene comprises or consists of a polynucleotide sequence that has at least 65, 70, 75, 80, 85, 90, 95, 96, 97, 98, 99 or 100% sequence identity with at least one of SEQ ID NO: 5 and SEQ ID NO: 25, of which SEQ ID NO: 5 is more preferred. More preferably, the regulatory element of the FOXA2 gene comprises or consists of a polynucleotide sequence that has at least 65, 70, 75, 80, 85, 90, 95, 96, 97, 98, 99 or 100% sequence identity with at least one of SEQ ID NO: 15 and SEQ ID NO: 35, of which SEQ ID NO: 15 is more preferred.

SOX17 (SRY-box 17) plays a key role in embryonic development where it is required for normal development of the definitive gut endoderm and looping of the embryonic heart tube. Furthermore, SOX17 plays an important role in embryonic and postnatal vascular development, including development of arteries. During embryonic differentiation, SOX17 increases over time, expression steadily increases and peaks around day 7, after which SOX17 expression slowly declines (see FIG. 1B). Disturbances in SOX17 expression can thus indicate that an agent thus has an effect on the intermediate stages of embryonic development.

Preferably, the regulatory element of the SOX17 gene comprises or consists of a polynucleotide sequence that has at least 65, 70, 75, 80, 85, 90, 95, 96, 97, 98, 99 or 100% sequence identity with at least one of SEQ ID NO: 6 and SEQ ID NO: 26, of which SEQ ID NO: 6 is more preferred. More preferably, the regulatory element of the SOX17 gene comprises or consists of a polynucleotide sequence that has at least 65, 70, 75, 80, 85, 90, 95, 96, 97, 98, 99 or 100% sequence identity with at least one of SEQ ID NO: 16 and SEQ ID NO: 36, of which SEQ ID NO: 16 is more preferred.

ALB (Albumin) is the main protein in plasma and produced by hepatocytes in the liver. hiPSC cells differentiated towards mature hepatocytes in vitro also produce and secrete Albumin. Albumin production is used as a hepatic activity and hepatocyte maturity measure. During embryonic differentiation towards hepatocytes, ALB expression increases over time, and peaks towards the end of the process (see FIG. 1B). Disturbances in ALB expression can thus indicate that an agent thus has an effect on the late stages of embryonic development.

Preferably, the regulatory element of the ALB gene comprises or consists of a polynucleotide sequence that has at least 65, 70, 75, 80, 85, 90, 95, 96, 97, 98, 99 or 100% sequence identity with at least one of SEQ ID NO: 7 and SEQ ID NO: 27, of which SEQ ID NO: 7 is more preferred. More preferably, the regulatory element of the ALB gene comprises or consists of a polynucleotide sequence that has at least 65, 70, 75, 80, 85, 90, 95, 96, 97, 98, 99 or 100% sequence identity with at least one of SEQ ID NO: 17 and SEQ ID NO: 37, of which SEQ ID NO: 17 is more preferred.

AFP (α-fetoprotein) is the fetal analog of albumin, also produced in hepatocytes and a major component of plasma during early embryonic development up to birth. After birth, levels of AFP decrease while levels of albumin increase. Hepatocytes differentiated in vitro from hiPSC can express both AFP and ALB towards the end of differentiation (Hannan et al, 2013). During embryonic differentiation towards hepatocytes, AFP expression increases over time, and peaks towards the end of the process (see FIG. 1B). Disturbances in AFP expression can thus indicate that an agent thus has an effect on the late stages of embryonic development

Preferably, the regulatory element of the AFP gene comprises or consists of a polynucleotide sequence that has at least 65, 70, 75, 80, 85, 90, 95, 96, 97, 98, 99 or 100% sequence identity with at least one of SEQ ID NO: 8 and SEQ ID NO: 28, of which SEQ ID NO: 8 is more preferred. More preferably, the regulatory element of the ALB gene comprises or consists of a polynucleotide sequence that has at least 65, 70, 75, 80, 85, 90, 95, 96, 97, 98, 99 or 100% sequence identity with at least one of SEQ ID NO: 18 and SEQ ID NO: 38, of which SEQ ID NO: 18 is more preferred.

Ck18 (cytokeratin 18) is a marker for mature hepatocytes. As differentiation proceeds, CK18 expression starts to emerge and peaks towards the end of the process (FIG. 7). Disturbances in Ck18 expression can thus indicate that an agent has an effect on the late stages of embryonic development

Preferably, the regulatory element of the Ck18 gene comprises or consists of a polynucleotide sequence that has at least 65, 70, 75, 80, 85, 90, 95, 96, 97, 98, 99 or 100% sequence identity with at least one of SEQ ID NO: 9 and SEQ ID NO: 29, of which SEQ ID NO: 29 is more preferred. More preferably, the regulatory element of the Ck18 gene comprises or consists of a polynucleotide sequence that has at least 65, 70, 75, 80, 85, 90, 95, 96, 97, 98, 99 or 100% sequence identity with at least one of SEQ ID NO: 19 and SEQ ID NO: 39, of which SEQ ID NO: 39 is more preferred.

Vegfr1 (Vascular endothelial growth factor receptor 1, FLT1) is a key player in the development of embryonic vasculature, the regulation of angiogenesis, cell survival, cell migration and macrophage function. As differentiation proceeds towards cardiomyocytes, Vegfr1 expression starts to emerge and peaks towards the end of the process (FIG. 6). Disturbances in Vegfr1 expression can thus indicate that an agent thus as an effect on the late stages of embryonic development

Preferably, the regulatory element of the Vegfr1 gene comprises or consists of a polynucleotide sequence that has at least 65, 70, 75, 80, 85, 90, 95, 96, 97, 98, 99 or 100% sequence identity with at least one of SEQ ID NO: 10 and SEQ ID NO: 30, of which SEQ ID NO: 30 is more preferred. More preferably, the regulatory element of the Vegfr1 gene comprises or consists of a polynucleotide sequence that has at least 65, 70, 75, 80, 85, 90, 95, 96, 97, 98, 99 or 100% sequence identity with at least one of SEQ ID NO: 20 and SEQ ID NO: 40, of which SEQ ID NO: 40 is more preferred.

In one embodiment of the invention, at least two reporter cell lines are contacted with the agent. Preferably, at least 3, 4, 5, 6, 7, 8, or 9 type of reporter cells lines are contacted with the agent. Preferably, when one or more reporter cell lines are contacted with the agent, the reporter cells line are contacted simultaneously with the agent.

In one embodiment of the invention, the type of reporter cell that is contacted with the agent in step b) comprises a regulatory element of the OCT4 gene as defined herein and one or more types of reporter cells each having a different regulatory element selected from the group consisting of:
a regulatory element of the BMP4 gene as defined herein;
a regulatory element of the MYH6 gene as defined herein;
a regulatory element of the PAX6 gene as defined herein;
a regulatory element of the FOXA2 gene as defined herein;
a regulatory element of the SOX17 gene as defined herein;
a regulatory element of the ALB gene as defined herein;
a regulatory element of the AFP gene as defined herein;
a regulatory element of the Ck18 gene as defined herein;
a regulatory element of the Vegfr1 gene as defined herein; and
a regulator element of the SOX1 gene as defined herein,

In one embodiment of the invention, the agent in step b) is contacted with a reporter cell comprising a regulatory element of the OCT4 gene as defined herein, a reporter cell comprising a regulatory element of the SOX1 gene as defined herein and with a reporter cell comprising a regulatory element of the PAX6 gene as defined herein. A combination of the OCT4, SOX1 and PAX6 reporter cells is used to follow hiPSC differentiation towards neural rosettes, where OCT4 expression can be used to monitor the loss of pluripotency, SOX1 marks the intermediate stage of development and PAX6 is used to follow the maturation of the neural rosettes.

In one embodiment of the invention, the agent in step b) is contacted with a reporter cell comprising a regulatory element of the OCT4 gene as defined herein, a reporter cell comprising a regulatory element of the BMP4 gene as defined herein, and a reporter cell comprising regulatory element of the MYH6 gene as defined herein. A combination of the OCT4, BMP4 and MYH6 can be used to follow hiPSC differentiating towards cardiomyocytes, where OCT4 expression can be used to monitor the loss of pluripotency, BMP4 marks the intermediate stage of development and MYH6 is used to follow the maturation of the cardiomyocytes.

In one embodiment of the invention, the agent in step b) is contacted with a reporter cell comprising a regulatory element of the OCT4 gene as defined herein, a reporter cell comprising a regulatory element of the FOXA2 gene as defined herein, a reporter cell comprising a regulatory element of the SOX17 gene as defined herein, and at least one of a reporter cell type comprising a regulatory element of the ALB gene as defined herein or a reporter cell type comprising a regulatory element of the AFP gene as defined herein. A combination of the OCT4, SOX17, FOXA2, and AFP or ALB can be used to follow hiPSC differentiating towards hepatocytes, where OCT4 expression can be used to monitor the loss of pluripotency, SOX17 and FOXA2 mark the intermediate stage of development and AFP or ALB is used to follow the maturation of the hepatocytes.

In preferred embodiments, present invention combines the differentiation of stem cells into the three embryonic cell lineages endoderm, mesoderm and ectoderm and subsequently into different functional tissues, in vitro mimicking the complex in vivo developmental processes. This integrated method, which preferably combines at least the seven biomarkers OCT4, FOXA2, BMP4, SOX1, AFP, MYH6 and PAX6, makes is possible to accurately identify chemicals that interfere with normal embryonic development, providing a comprehensive battery approach. Validation of the method confirms that integration of these seven biomarkers for endoderm, mesoderm and ectoderm and mature liver, heart, and neural cells is required to accurately identify chemicals that are classified as teratogenic in humans. FIG. 9 summarizes the correlation of the in vivo classification of five well-established teratogenic and one non-teratogenic compounds with their respective in vitro prediction based on the method as described herein that utilizes the at least the seven biomarkers OCT4, FOXA2, BMP4, SOX1, AFP, MYH6 and PAX. This method allows the evaluation of the specific biomarker expression targeting liver, heart and neural tissues, both in early and late stages of lineage differentiation. Expression of the biomarker OCT4 is decreased upon differentiation of the pluripotent stem cells, while early developmental markers FOXA2, BMP4 and SOX1 are expressed during establishment of endoderm, mesoderm and ectoderm, respectively. Upon further maturation of the liver, heart and neural tissues, expression of the early lineage biomarkers is reduced and activation of the liver-specific biomarker AFP, cardiomyocyte biomarker MYH6 and neural biomarker PAX6 is observed. Alterations in the expression pattern of one or more of these seven biomarkers after chemical exposure indicate perturbation of stem cell differentiation and early embryonic development, indicating the teratogenic properties of the tested agent. FIG. 9 table illustrates the importance of combining the seven biomarkers to make it possible to assay the main primary cell lineages: endo-, meso- and ectoderm, and proves the power of the methods predictability, which is precisely correlated with the in vivo classification of the respective compounds.

Accordingly, in one preferred embodiment of the invention, at least seven reporter cell types are contacted with the agent in step b) each of the at least seven reporter cell lines comprising a different regulatory element selected from:
a regulatory element of the OCT4 gene as defined herein.
a regulatory element of the BMP4 gene as defined herein;
a regulatory element of the MYH6 gene as defined herein; and
a regulatory element of the PAX6 gene as defined herein;

and at least one of:
a regulatory element of the FOXA2 gene as defined herein;
a regulatory element of the AFP gene as defined herein; or
a regulatory element of the SOX1 gene as defined herein.

In one embodiment, the reporter sequence according to the invention is a gene encoding a protein. Preferably, a reporter sequence encodes a protein that is readily detectable either by its presence, its association with a detectable moiety or by its activity that results in the generation of a detectable signal. In certain aspects, a detectable moiety may include a radionuclide, a fluorophore, a luminophore, a microparticle, a microsphere, an enzyme, an enzyme substrate, a polypeptide, a polynucleotide, a nanoparticle, and/or a nanosphere, all of which may be coupled to an antibody or a ligand that recognizes and/or interacts with a reporter. Generally, although not necessarily, the reporter sequence includes a nucleic acid sequence and/or encodes a detectable polypeptide that are not otherwise produced by the cells. Many reporter genes have been described and are available from a variety of sources including commercial sources such as, e.g., BioVision, EMD Millipore, Invitrogen, amongst other sources. Signals that may be detected include, but are not limited to color, fluorescence, luminescence, isotopic or radio-isotopic signals, cell surface tags, cell viability, relief of a cell nutritional requirement, cell growth and drug resistance. Reporter sequences include, but are not limited to, DNA sequences encoding .beta.-lactamase, .beta.-galactosidase (LacZ), alkaline phosphatase, thymidine kinase, green fluorescent protein (GFP), chloramphenicol acetyltransferase (CAT), luciferase, membrane bound proteins including, for example, G-protein coupled receptors (GPCRs), somatostatin receptors, CD2, CD4, CD8, the influenza hemagglutinin protein, symporters (such as NIS) and others well known in the art, to which high affinity antibodies or ligands directed thereto exist or can be produced by conventional means,

In one embodiment, a reporter sequence encodes a fluorescent protein. Examples of fluorescent proteins which may be used in accord with the invention include green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine, mCherry and red fluorescent protein from discosoma (dsRED). It is to be understood that these examples of fluorescent proteins are not exclusive and may encompass later developed fluorescent proteins, such as any fluorescent protein within the infrared, visible or ultraviolet spectra. Even more preferably, the fluorescent protein is green fluorescent protein (GFP), mCherry. DsRed or derivatives thereof. Most preferably, the fluorescent protein is SEQ ID NO: 41 or SEQ ID NO: 100.

In one embodiment of the invention, the reporter cell comprises a regulatory element of the OCT4 gene as described herein and a reporter sequence that comprises or consists of SEQ ID NO: 100.

In one embodiment of the invention, the reporter cell comprises a regulatory element of the BMP4 gene as described herein and a reporter sequence that comprises or consists of SEQ ID NO: 41.

In one embodiment of the invention, the reporter cell comprises a regulatory element of the MYH6 gene as described herein and a reporter sequence that comprises or consists of SEQ ID NO: 41.

In one embodiment of the invention, the reporter cell comprises a regulatory element of the PAX6 gene as described herein and a reporter sequence that comprises or consists of SEQ ID NO: 41.

In one embodiment of the invention, the reporter cell comprises a regulatory element of the FOX2A gene as described herein and a reporter sequence that comprises or consists of SEQ ID NO:41.

In one embodiment of the invention, the reporter cell comprises a regulatory element of the SOX17 gene as described herein and a reporter sequence that comprises or consists of SEQ ID NO:41.

In one embodiment of the invention, the reporter cell comprises a regulatory element of the ALB gene as described herein and a reporter sequence that comprises or consists of SEQ ID NO:41.

In one embodiment of the invention, the reporter cell comprises a regulatory element of the AFP gene as described herein and a reporter sequence that comprises or consists of SEQ ID NO:41.

In one embodiment of the invention, the reporter cell comprises a regulatory element of the Ck18 gene as described herein and a reporter sequence that comprises or consists of SEQ ID NO:41.

In one embodiment of the invention, the reporter cell comprises a regulatory element of the Vegfr1 gene as described herein and a reporter sequence that comprises or consists of SEQ ID NO:41.

In one embodiment of the invention, the reporter cell comprises a regulatory element of the SOX1 gene as described herein and a reporter sequence that comprises or consists of SEQ ID NO:41.

In another preferred embodiment, the reporter sequence is selected from the group consisting of horse radish peroxidise (HRP), luciferase, chloramphenicol acetyl transferase (CAT) and β-galactosidase.

The agent can be any type of compound of composition comprising a compound. For example, the agent can be a pharmaceutical agent, a chemical agent, an agrochemical agent, a cosmetic, a plasticizer, a dye or a food-ingredient. In one embodiment, the agent is an agent with suspected teratogenic toxicity.

In one embodiment, the agent is a polypeptide, a peptide, a nucleic acid, a (small) molecule, or a natural product.

In a second aspect, the invention provides for a kit of parts comprising:
one or more types of reporter cell, wherein each type of reporter cell comprises a reporter sequence operatively linked to a regulatory element of a gene and wherein the one or more type of reporter cells each comprise a different regulatory element selected from:
a regulatory element of the OCT4 gene as defined herein;
a regulatory element of the BMP4 gene as defined herein;
a regulatory element of the MYH6 gene as defined herein;
a regulatory element of the PAX6 gene as defined herein;
a regulatory element of the FOXA2 gene as defined herein;
a regulatory element of the SOX17 gene as defined herein;
a regulatory element of the ALB gene as defined herein;
a regulatory element of the AFP gene as defined herein;
a regulatory element of the Ck18 gene as defined herein;
a regulatory element of the Vegfr1 gene as defined herein; and
a regulatory element of the SOX1 gene as defined herein.

In one embodiment, the kit of parts comprises at least 2, 3, 4, 5, 6, 7, 8 or 9 types of reporter cells.

In one embodiment the kit of parts comprises a reporter cell comprising a regulatory element of the OCT4 gene as defined herein, a reporter cell comprising a regulatory element of the SOX1 gene as defined herein and a reporter cell comprising a regulatory element of the PAX6 gene as defined herein.

In one embodiment the kit of parts comprises a reporter cell comprising a regulatory element of the OCT4 gene as defined herein, a reporter cell comprising a regulatory element of the BMP4 gene as defined herein, and a reporter cell comprising a regulatory element of the MYH6 gene as defined herein.

In one embodiment the kit of parts comprises a reporter cell comprising a regulatory element of the OCT4 gene as defined herein, a reporter cell comprising a regulatory element of the FOXA2 gene as defined herein, a reporter cell comprising a regulatory element of the SOX17 gene as defined herein and at least one of a reporter cell type comprising a regulatory element of the ALB gene as defined herein or a reporter cell type comprising a regulatory element of the AFP gene as defined herein.

In a preferred embodiment, the kit of parts consists of a reporter cell comprising a regulatory element of the OCT4 gene as defined herein, a reporter cell comprising a regulatory element of the BMP4 gene as defined herein, a reporter cell comprising a regulatory element of the MYH6 gene as defined herein, a reporter cell comprising a regulatory element of the FOXA2 gene as defined herein, a reporter cell comprising a regulatory element of the AFP gene as defined herein, a reporter cell comprising a regulatory element of the PAX6 gene as defined herein, a reporter cell comprising a regulatory element of the SOX1 gene as defined herein.

In a third aspect, the invention provides for a combination of at least two transgenic non-human mammals comprising at least one cell comprising a reporter sequence operatively linked to a different regulatory element and wherein the regulatory element is selected from:
a regulatory element of the OCT4 gene as defined herein;
a regulatory element of the BMP4 gene as defined herein;
a regulatory element of the MYH6 gene as defined herein;
a regulatory element of the PAX6 gene as defined herein;
a regulatory element of the FOXA2 gene as defined herein;
a regulatory element of the SOX17 gene as defined herein;
a regulatory element of the ALB gene as defined herein;
a regulatory element of the AFP gene as defined herein;
a regulatory element of the Ck18 gene as defined herein;
a regulatory element of the Vegfr1 gene as defined herein; and
a regulatory element of the SOX1 gene as described herein.

Preferably, the transgenic non-human animals are rodents such as mice, rats, guinea pigs, hamsters and gerbils. The transgenic non-human animal that is most preferred is mice.

In one embodiment of the invention, the combination or non-human transgenic animals comprises at least 3, preferably at least, 4, 5, 6, 7 8, or 9 transgenic non-human mammals.

In one preferred embodiment, the invention provides for a combination of at least seven transgenic non-human mammals comprising at least one cell comprising a reporter sequence operatively linked to a different regulatory element, wherein the at least at least seven transgenic non-human mammals comprise a cell comprising a regulatory element of the OCT4 gene as defined herein, a regulatory element of the BMP4 gene as defined herein, a regulatory element of the MYH6 gene as defined herein, a regulatory element of the PAX6 gene as defined herein, a regulatory element of the FOXA2 gene as defined herein, a regulatory element of the AFP gene as defined herein and a regulatory element of the SOX1 gene as described herein.

In a forth aspect the invention provides for a use of one or more types of reporter cells for the determination of an effect of an agent on mammalian embryonic development,

wherein each type of reporter cell comprises a reporter sequence operatively linked to a different regulatory element of a gene,

and wherein the different regulatory elements for each type of reporter cell is selected from the group consisting of:
a regulatory element of the OCT4 gene as defined herein;
a regulatory element of the BMP4 gene as defined herein;
a regulatory element of the MYH6 gene as defined herein;
a regulatory element of the PAX6 gene as defined herein;
a regulatory element of the FOXA2 gene as defined herein;
a regulatory element of the SOX17 gene as defined herein;
a regulatory element of the ALB gene as defined herein;
a regulatory element of the AFP gene as defined herein;
a regulatory element of the Ck18 gene as defined herein;
a regulatory element of the Vegfr1 gene as defined herein; and
a regulatory element of the SOX1 gene as described herein.

In a fifth aspect, the invention provides for a reporter cell as defined herein. In one embodiment, the invention provides for a collection of reporter cells as defined herein. In one embodiment, the collection of reporter cells comprises at least two reporter cells, at least 3, 4, 5, 6, 7, 8 or 9 reporter cells. In one preferred embodiment, the collection of reporter cells comprises at least seven reporter cells comprising a reporter cell comprising the regulatory element of OCT4 as herein described, a reporter cell comprising the regulatory element of BMP4 as herein described, a reporter cell comprising the regulatory element of MYH6 as herein described, a reporter cell comprising the regulatory element of FOXA2 as herein described, a reporter cell comprising the regulatory element of AFP as herein described, a reporter cell comprising the regulatory element of SOX1 as herein described, a reporter cell comprising the regulatory element of PAX6 as herein described,

### Definitions

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using global alignment algorithms (e.g. Needleman Wunsch) which align the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall lengths, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.hih.gov/.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer.

Any reference to nucleotide or amino acid sequences accessible in public sequence databases herein refers to the version of the sequence entry as available on the filing date of this document.

All patent and literature references cited in the present specification are hereby incorporated by reference in their entirety.

### DESCRIPTION OF THE FIGURES

FIG. 1: hiPSC cells were differentiated towards cardiomyocytes, hepatocyte and neural rosettes by addition of specific growth factors at specific times during differentiation (as described herein). RNA samples were collected at Day 0, Day 7 and Day 10, 14 or 21 and biomarker expression was analyzed using qPCR.

A) Cardiomyocyte differentiation. OCT4 was expressed in pluripotent stem cells and during differentiation the expression decreased. BMP4 marked the middle stage of the differentiation and expression peaks around day 7. MYH6 was expressed in mature cardiomyocytes and expression increased overtime.

B) Hepatocyte differentiation. OCT4 was expressed in pluripotent stem cells and during differentiation the expression decreased. SOX17 and FOXA2 were expressed around day 7 which marks the intermediate stage of differentiation. ALB and AFP were expressed during late stages of differentiation.

C) Neural rosette formation. OCT4 was expressed in pluripotent stem cells and during differentiation the expression decreases. SOX1 marked the middle stage of the differentiation and expression peaks around day 7. PAX6 is expressed in neural rosettes and expression increases over time.

FIG. 2: Differentiation in the presence of retinoic acid. To measure the effect of the teratogenic compound retinoic acid, hiPSC cells were differentiated towards hepatocytes (A), cardiomyocytes (B) and neural rosettes (C) in the presence of retinoic acid. During and at the end of differentiation, RNA samples were collected and biomarker expression was analyzed using qPCR. * p<0.05 in T-test compared to vehicle control.

FIG. 3: Differentiation in the presence of acrylamide. To measure the effect of the non-teratogenic compound acrylamide, hiPSC cells were differentiated towards hepatocytes (A) and cardiomyocytes (B) in the presence of acrylamide. During and at the end of differentiation, RNA samples were collected and biomarker expression was analyzed using qPCR. * p<0.05 in T-test compared to vehicle control (no changes were significant).

FIG. 4: OCT4-GFP reporter cell line. hiPSC were genetically modified to express OCT4-GFP from the endogenous locus. HiPSC express OCT4 in the pluripotent state (Day 0). (A) FACS plots show the GFP intensity of wild-type unmodified hiPSC and OCT4-GFP hiSPC cells. (B) percentage of GFP positive cells and GFP intensity of wild-type and OCT-GFP hiPSC (clone 1) during differentiation towards cardiomyocytes.

FIG. 5: Defective cardiomyocyte differentiation upon teratogenic compound exposure. mES cells were exposed to teratogenic compounds during mES cell differentiation towards cardiomyocytes. The percentage of beating 3D embryoid bodies (examples in A), representing functional cardiomyocytes, was counted. (B) Quantification of beating bodies. Presented values are expressed as percentages of control. Students t-test was used to determine significance. *p<0.05, **p<0.01, ***p<0.001.

FIG. 6: mES cells were differentiated towards cardiomyocytes or hepatocytes by the addition of specific growth factors at specific times during differentiation (as described herein). RNA samples were collected at Day 0, Day 5, Day 12 and Day 18 and biomarker expression was analyzed using qPCR.

A) Cardiomyocyte differentiation. OCT4 was expressed in pluripotent mES cells and expression decreased as differentiation progressed. BMP4 was expressed at the intermediate stage of differentiation and expression peaked around day 5. Vegfr1 marked maturing cardiomyocytes and expression peaked around day 12.

B) Hepatocyte differentiation. OCT4 was expressed in pluripotent mES cells and expression decreased as differentiation progressed. FOXA2 and SOX17 were expressed at the intermediate stage of differentiation and expression peaks around day 10.

FIG. 7: GFP-Ck18 reporter cells were differentiated towards hepatocytes. During differentiation, the expression of GFP-CK18, a marker for mature hepatocytes, increased overtime, which can be observed using microscopy (A). (B) Expression of GFP-CK18 was measured by quantifying the GFP expression. GFP-intensity calculated with Fiji image processing package.

FIG. 8: Defective hepatocyte differentiation upon teratogenic compound exposure. mES GFP-Ck18 reporter cells were exposed to different teratogenic compounds during mES cell differentiation towards hepatocytes. After 21 days, fluorescence intensity of the hepatocyte specific GFP-Ck18 reporter was measured using microscopy and quantified using Fiji image processing software. GFP intensity was normalized to nuclear intensity measured using DAPI. Presented values are expressed as percentages of control. Student t-test was used for determination of significance. *p<0.05 **p<0.01.*** p<0.001.

FIG. 9: Table summarizing the correlation of the in vivo classification of 5 well-established teratogenic and 1 non-teratogenic compounds with their respective in vitro prediction based method as described herein which utilizes seven different types of reporter cells as indicated in the table. Reduction (+) of the biomarker expression means disruption of the developmental processes, which flags the teratogenic potential of the respective compound under assessment. No effect on the biomarker expression (−) indicates the non-teratogenic compounds. Inconclusive results have been additionally highlighted (i).

### DESCRIPTION OF THE SEQUENCES

TABLE 1
Sequences

| SEQ ID NO: | Name |
|---|---|
| 1 | OCT4 promotor region (human) |
| 2 | BMP4 promotor region (human) |
| 3 | MYH6 promotor region (human) |
| 4 | PAX6 promotor region (human) |
| 5 | FOXA2 promotor region (human) |
| 6 | SOX17 promotor region (human) |
| 7 | ALB promotor region (human) |
| 8 | AFP promotor region (human) |
| 9 | Ck18 promotor region (human) |
| 10 | Vegfr1 promotor region (human) |
| 11 | OCT4 regulatory element (human) |
| 12 | BMP4 regulatory element (human) |
| 13 | MYH6 regulatory element (human) |
| 14 | PAX6 regulatory element (human) |
| 15 | FOXA2 regulatory element (human) |
| 16 | SOX17 regulatory element (human) |
| 17 | ALB regulatory element (human) |
| 18 | AFP regulatory element (human) |
| 19 | Ck18 regulatory element (human) |
| 20 | Vegfr1 regulatory element (human) |
| 21 | OCT4 promotor region (mouse) |
| 22 | BMP4 promotor region (mouse) |
| 23 | MYH6 promotor region (mouse) |
| 24 | PAX6 promotor region (mouse) |
| 25 | FOXA2 promotor region (mouse) |
| 26 | SOX17 promotor region (mouse) |
| 27 | ALB promotor region (mouse) |
| 28 | AFP promotor region (mouse) |
| 29 | Ck18 promotor region (mouse) |
| 30 | Vegfr1 promotor element (mouse) |
| 31 | OCT4 regulatory element (mouse) |
| 32 | BMP4 regulatory element (mouse) |
| 33 | MYH6 regulatory element (mouse) |
| 34 | PAX6 regulatory element (mouse) |
| 35 | FOXA2 regulatory element (mouse) |
| 36 | SOX17 regulatory element (mouse) |
| 37 | ALB regulatory element (mouse) |
| 38 | AFP regulatory element (mouse) |
| 39 | Ck18 regulatory element (mouse) |
| 40 | Vegfr1 regulatory element (mouse) |
| 41 | GFP with PGK-NEO selection (N-terminal) |
| 42 | hOCT4_BAC_GFP_FW |
| 43 | hOCT4_BAC_GFP_REV |
| 44 | hALB_BAC_GFP-N-F |
| 45 | hALB_BAC_GFP-N-R |
| 46 | hAFP_BAC_GFP-N_F |
| 47 | hAFP_BAC_GFP-N_R |
| 48 | hMYH6_BAC_GFP-N_F |
| 49 | hMYH6_BAC_GFP-N_R |
| 50 | mCK18_BAC_GFP_NF |
| 51 | mCK18_BAC_GFP_NR |
| 52 | hOCT4_homDonor_F |
| 53 | hOCT4_homDonor_R |
| 54 | hALB_Hom_N_FW |
| 55 | hALB_Hom_N_REV |
| 56 | hAFP_Hom_N_FW |
| 57 | hAFP_Hom_N_REV |
| 58 | hMYH6_Hom_N_FW |
| 59 | hMYH6_Hom_N_REV |
| 60 | hOct4-Crispr_F |
| 61 | hOct4-Crispr-R |
| 62 | ALB-Cripsr-N1 |
| 63 | ALB-Cripsr-N1 |
| 64 | AFP-Crispr-N1 |
| 65 | AFP-Crispr-N1 |
| 66 | MYH6-Crispr-N1 |
| 67 | MYH6-Crispr-N1 |
| 68 | hBMP4-FW2 |
| 69 | hBMP4-Rev2 |
| 70 | hSOX17-FW2 |
| 71 | hSOX17 Rev2 |
| 72 | hAFP-FW3 |
| 73 | hAFP-Rev3 |
| 74 | hALB-FW3 |
| 75 | hALB-Rev3 |
| 76 | GAPDH-human-F |
| 77 | GAPDH-human-R |
| 78 | Hprt-human-F |
| 79 | Hprt-human-R |
| 80 | MYH6-FW |
| 81 | MYH6-Rev |
| 82 | FoxA2-human-F |
| 83 | FoxA2-human-R |
| 84 | Oct-4-human-F |
| 85 | Oct4-human-R |
| 86 | Hprt-F |
| 87 | Hprt-R |
| 88 | GAPDH-F |
| 89 | GAPDH-R |
| 90 | Oct4 |
| 91 | Oct4 |
| 92 | BMP-4 |
| 93 | BMP-4 |
| 94 | SOX17 |
| 95 | SOX17 |
| 96 | FOXA2 |
| 97 | FOXA2 |
| 98 | VEGFR |
| 99 | VEGFR |
| 100 | GFP with PGK-NEO selection (C-terminal) |
| 101 | SOX1 promotor region (human) |
| 102 | SOX1 regulatory region (human) |
| 103 | SOX1 promotor region (mouse) |
| 104 | SOX1 regulatory region (mouse) |
| 105 | SOX1_FW |
| 106 | SOX1_Rev |

### EXAMPLES

Materials and Methods

HiPSC Cell Culture

Human Episomal iPSC Line (hiPSC) obtained from Thermo Scientific (A18945) were passaged as clumps using ReleasR and maintained in mTESR medium (StemCell Technologies) with 0.5% PS on Matrigel (Corning) according to established protocols.

mES Cell Culture

C57/Bl6 B4418 wild type mouse ES (mES) cells were cultured in ES knockout medium (Gibco) containing 10% FCS, 2 mM glutamax, 1 mM sodium pyruvate, 1% non-essential amino acids (NEAA), 1% pencillin/streptomycin (PS), 0.1 mM 2-mercaptoethanol and leukemia inhibitory factor (LIF) as previously described (Hendriks et al., 2016). Mouse ES cells were propagated on irradiated primary mouse embryonic fibroblasts as feeders according to established protocols.

### Example 1: Human iPSC Differentiation

For differentiation, single cell suspensions were created using Tryple select (Thermo) and single cell suspensions were counted using a flow cytometer before seeding.

Cardiomyocyte

The protocol for cardiomyocyte differentiation is based on (Den Hartogh et al., 2015, which is incorporated herein by reference). Briefly, on day −4, hiPSC cells were seeded in 24-well plates in mTESR medium on Matrigel. On day 0, the medium was replaced with BEL medium (IMDM, HF12 medium, PFHMII medium, BSA, ITS-X, CD lipids, α-Monothioglycerol, Glutamax, PS and ascorbic acid) containing BMP4, Activin A and Chir99021. At day 3 of differentiation, the medium was refreshed with BEL containing Xav939. On day 7 and 10 of differentiation, the medium was replaced with BEL without growth factors. RNA samples were collected on day 0, day 7 and day 14 of differentiation.

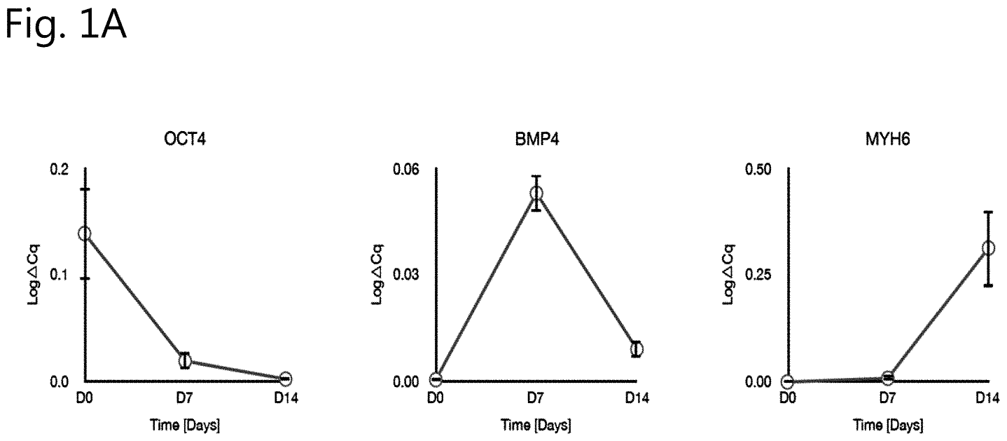

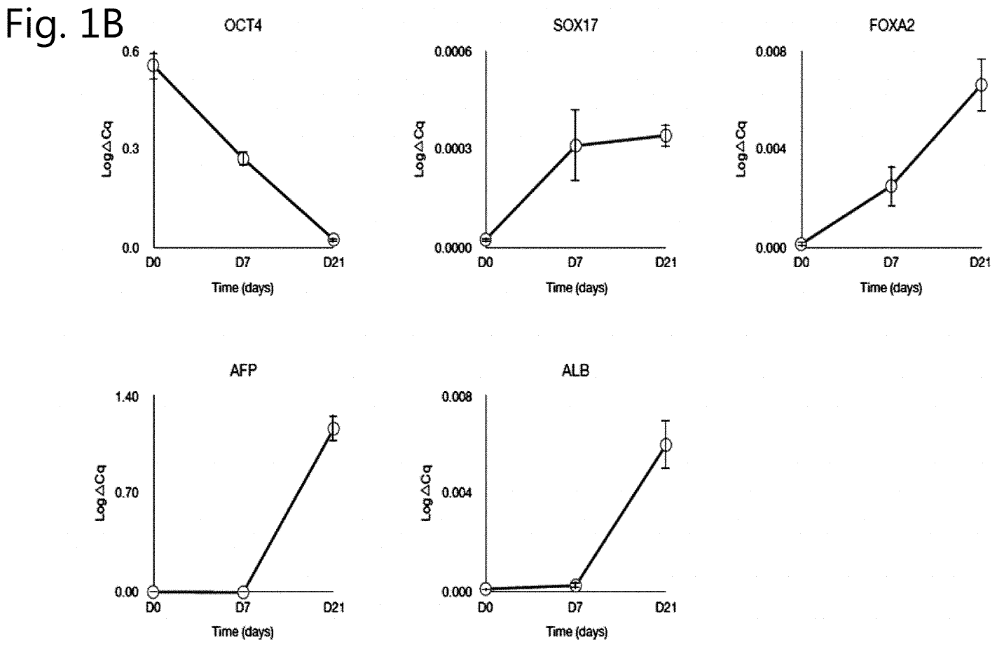

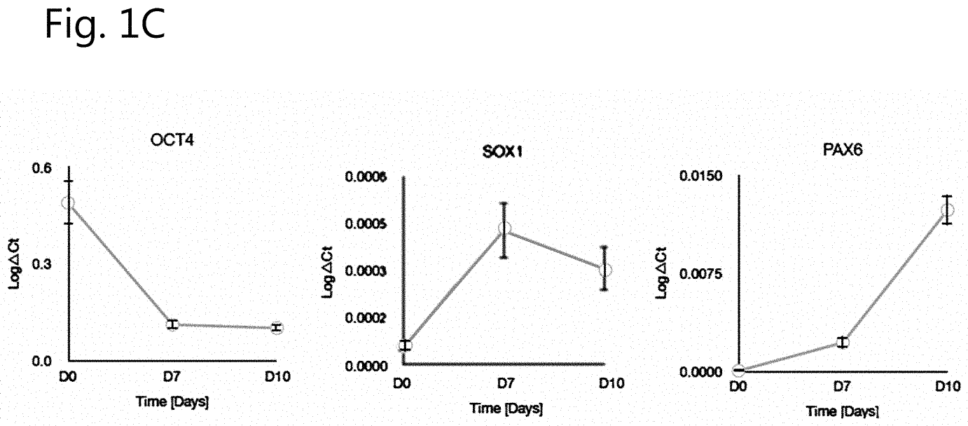

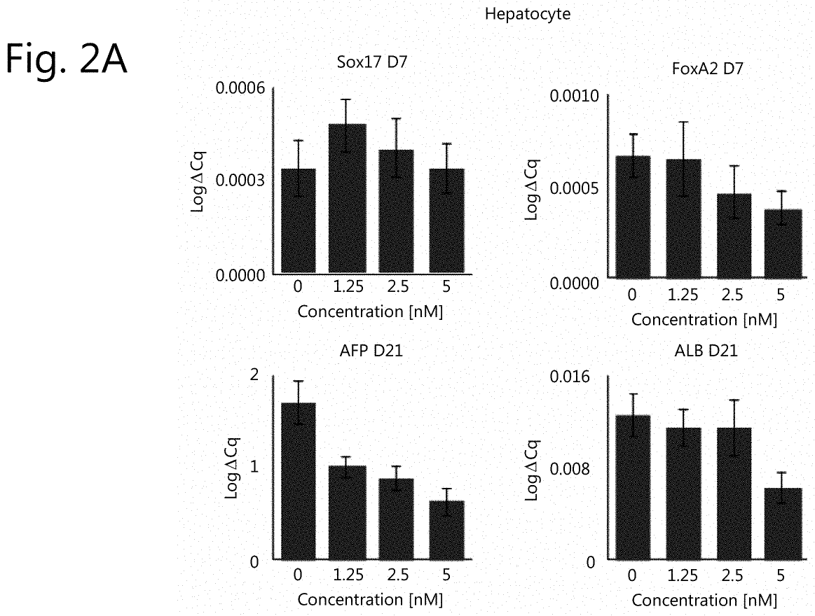

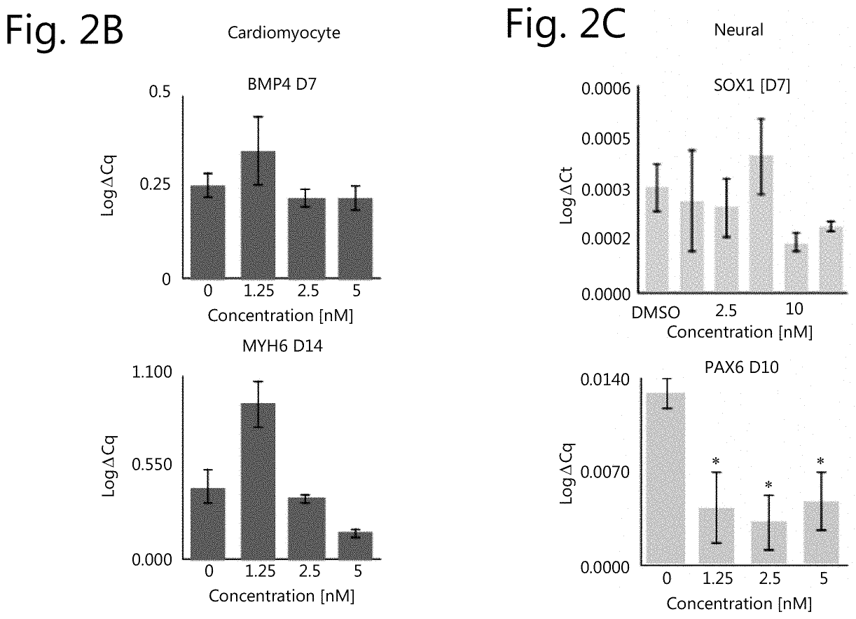

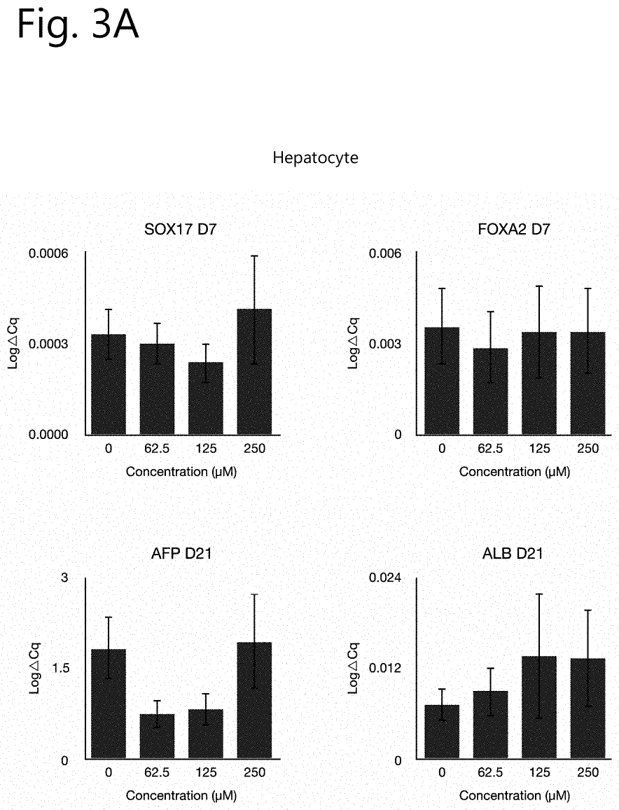

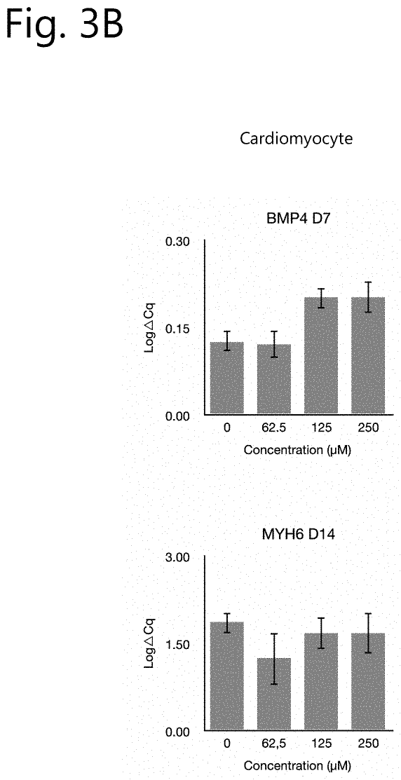

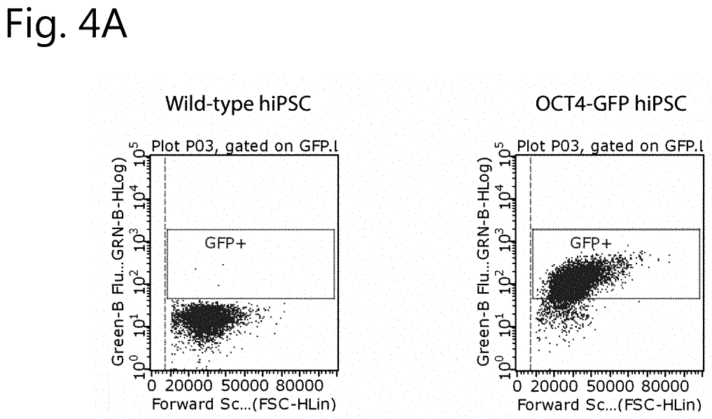

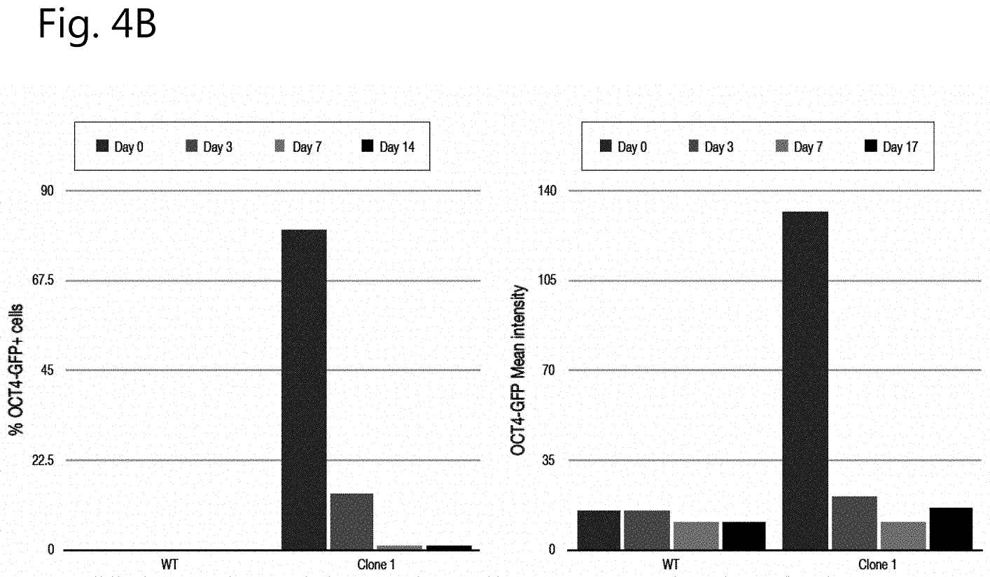

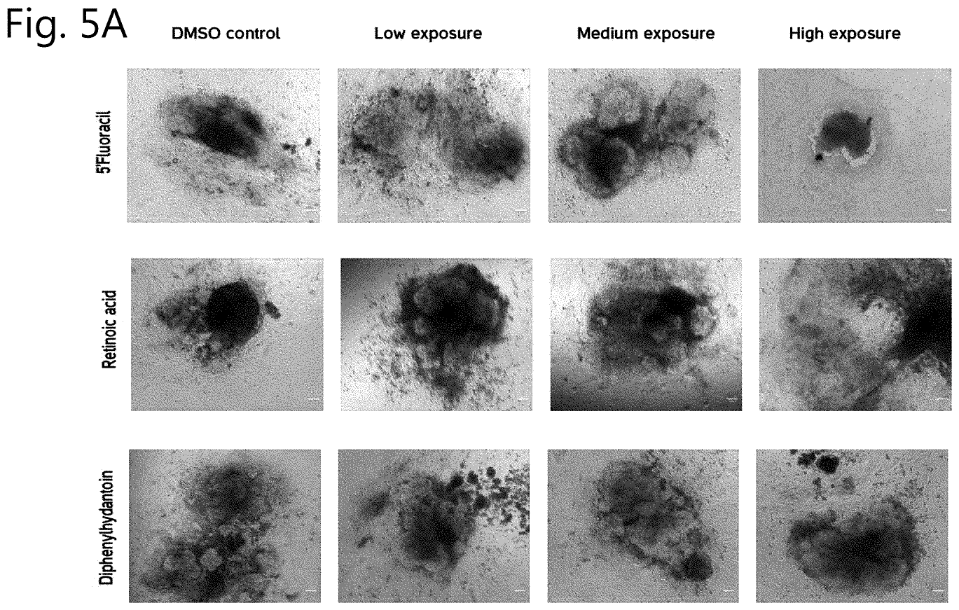

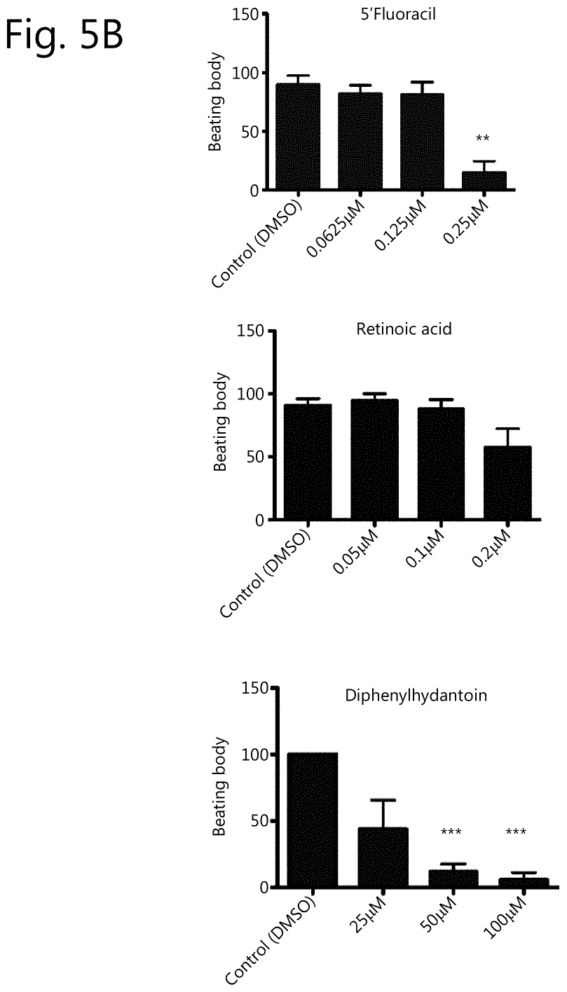

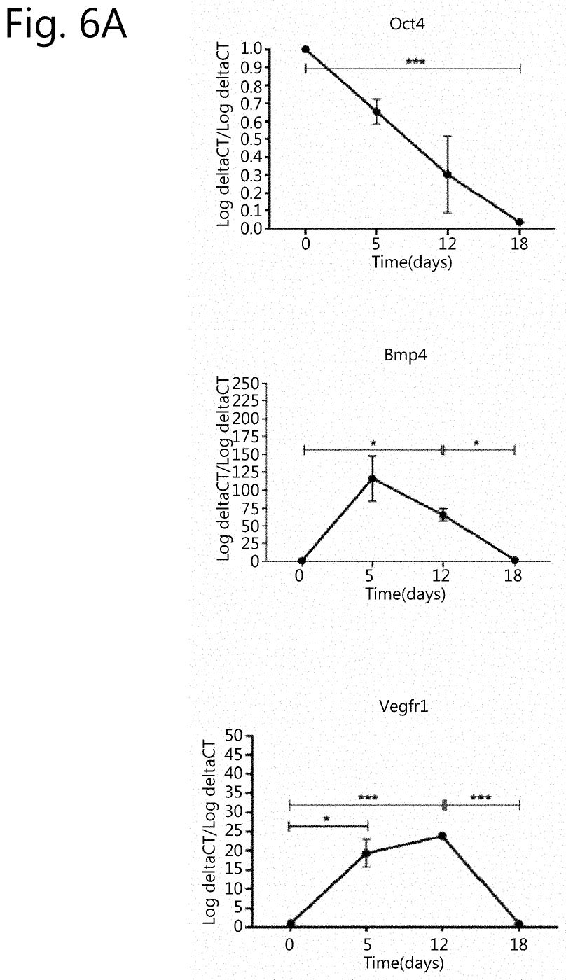

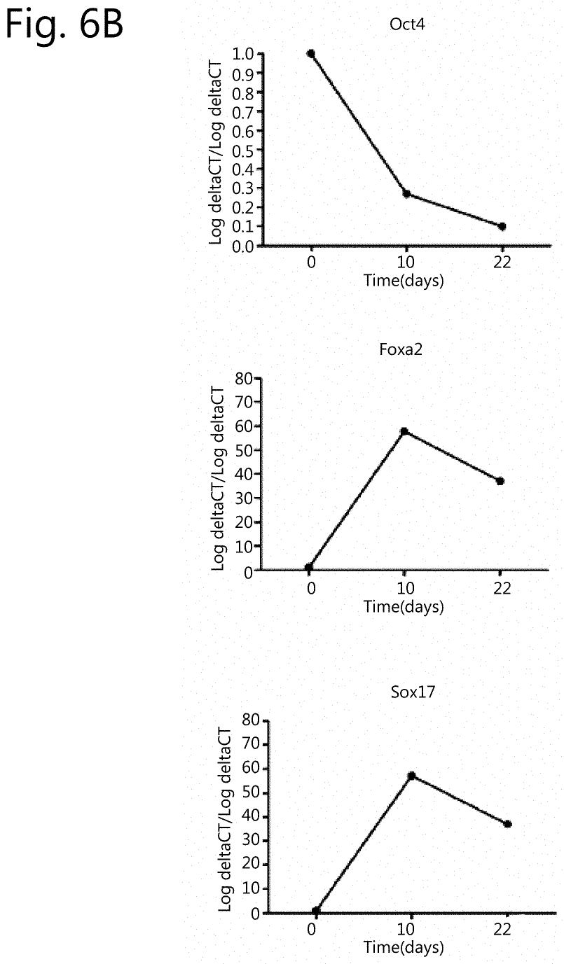

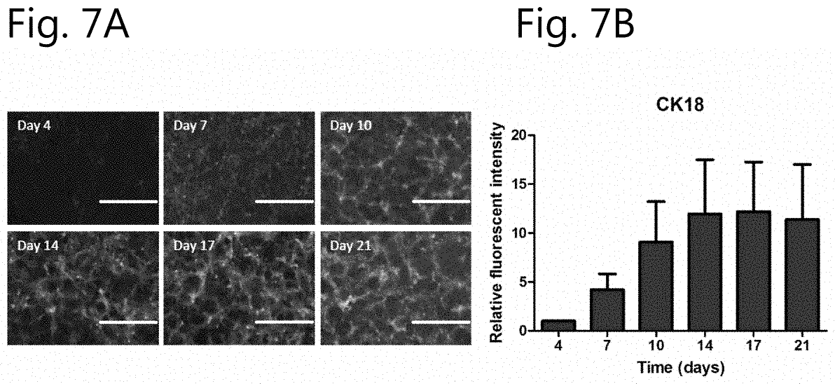

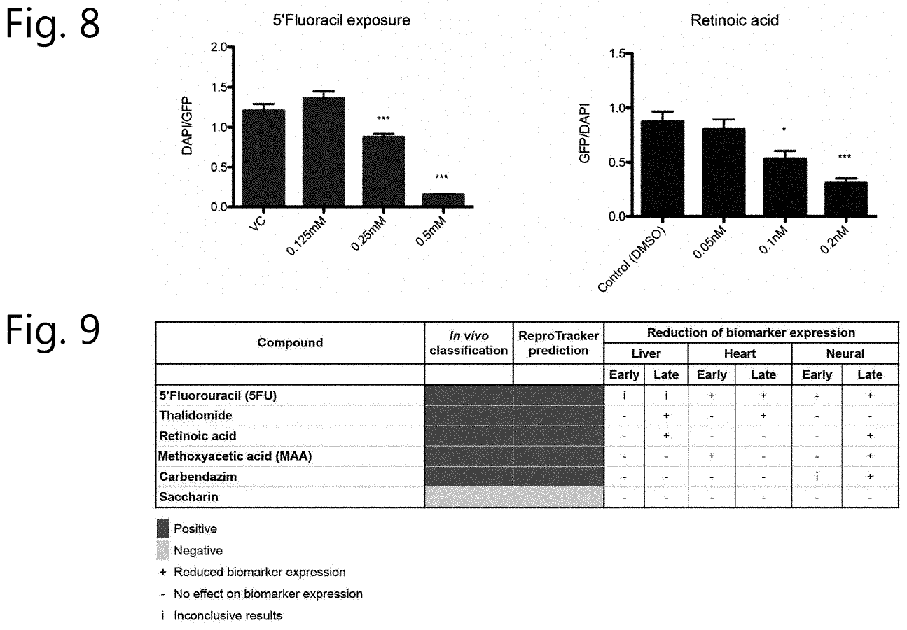

The results can be seen in FIG. 1A: OCT4, a marker of pluripotency was expressed at day 0 and, as expected, the expression decreased during differentiation. It was further found that BMP4 is a good marker for the middle stage of differentiation as the expression peaks around day 7, while MYH6 was found to be a good marker for mature cardiomyocytes, its expression increasing over time.

Hepatocyte

The hepatocyte differentiation protocol is based on (Chen et al., 2012, which is incorporated herein by reference). In brief, hiPSC cells were seeded on day −4 in mTESR medium on Matrigel. On day 0 the mTESR medium was replaced with RPMI medium containing B27, Activin A, Chir99021, HGF and PS. On day 3, the cells were refreshed with DMEM medium containing KO serum replacement, Glutamax, NEAA, 2-mercaptoethanol, DMSO and PS. On day 7, the medium was replaced with IMDM medium containing oncostatin M, dexamethasone, ITS, Glutamax, NEAA and PS. On day 10, 14 and 17, cells are refreshed with IMDM medium containing ITS, Glutamax, NEAA and PS. RNA samples were collected on day 0, day 7 and day 21 of differentiation.

The results can be seen in FIG. 1B: OCT4, a marker of pluripotency was expressed at day 0 and, as expected, the expression decreased during differentiation. SOX17 and FOXA2 were expressed around day 7 and can therefore be used to test whether differentiation is affected at the intermediate stage. ALB and AFP were expressed during late stages of differentiation and these markers were used to see whether differentiation is affected at the late stage.

Neural Rosette

The protocol for neural differentiation is based on (Lippmann, Estevez-Silva, & Ashton, 2014, which is incorporated herein by reference). In short, hiPSC were seeded in mTESR medium on Matrigel on day −1. On day 0, medium was replaced with E6 medium with PS to start differentiation. Medium was refreshed with E6 medium on Day 3 and 7. RNA samples were collected on day 0, day 7 and day 10.

The results can be seen in FIG. 1C: OCT4, a marker of pluripotency, is expressed at Day 0 and, as expected, the expression decreases during differentiation. SOX1 is expressed around day 7 and can therefore be used to test whether differentiation is affected at the intermediate stage. PAX6 is expressed in maturing neural rosettes and thus expression increased overtime.

### Example 2: Effect of Teratogenic Compounds on hiPSCs

Compound Exposure During Differentiation

To assess the effect of potential teratogenic agents, hiPSC were treated with the test material from day 0 until the end of differentiation. When differentiation medium was refreshed, fresh compound was added. The maximum concentration of the vehicle was 0.1% for DMSO and All-trans retinoic acid, acrylamide, 5-fluorouracil, diphenylhydantoin and thalidomide were dissolved in DMSO.

RNA Isolation, cDNA Synthesis and qPCR

Induction of the biomarker expression was compared with the expression of the gene in undifferentiated cells using quantitative real-time PCR (qRT-PCR). At several time points during differentiation, total RNA was isolated using Trizol (Qiagen). Complementary DNA was synthesized using oligo(dT) primers and SuperScript VI ReverseTranscriptase (Invitrogen) according to the manufacturer's protocol. Expression of biomarker genes was determined using specific primers (SEQ ID NOs: 68-99, 105 and 106) spanning the exon-exon boundaries of the genes with the PowerUP SYBR Green Master Mix (Applied Biosystems) on a Quantstudio 5 Real-Time PCR System (Applied Biosystems) using ROX as a passive reference. Relative expression was normalized using expression of the GAPDH and HPRT genes.

Results

To measure the effect of the teratogenic compound retinoic acid, hiPSC cells were differentiated towards hepatocytes, cardiomyocytes and neural rosettes in the presence of retinoic acid as described here above. Retinoic acid is a known teratogenic compound and as such, this allowed us to measure whether or our selected biomarkers would reflect the teratogenic nature of retinoic acid. Expression of AFP and ALB in hepatocytes, MYH6 in cardiomyocytes and PAX6 in neural rosettes was reduced when retinoic acid was added during differentiation (see FIG. 2).

In contrast, when hiPSC cells were differentiated to hepatocytes and cardiomyocytes in the presence of the non-teratogenic compound, acrylamide, no significant reduction of biomarker expression was observed during hepatocyte or cardiomyocyte differentiation (see FIG. 3).

### Example 3: OCT4-GFP Reporter Cell Line

Generation of GFP Reporter Cell Lines

The constructs for the GFP reporters were generated by BAC recombineering as described (Poser et al., 2008). Bacterial strains with a BAC containing the biomarker gene were selected using the mouse or human BAC finder and ordered from Thermo Scientific. The putative biomarker genes on the BAC were modified with a N-terminal or C-terminal GFP green fluorescent marker (Poser et al., 2008, supra) using BAC recombineering (SEQ ID NOs: 41,100). Electrocompetent BAC strains were first transformed with the pRed/ET plasmid that contains the RecE and RecT recombination enzymes. In the N-terminal GFP cassette, GFP consists of two exons, which are separated by the PGK promotor and neomycin selection cassette in the intron. The C-terminal GFP cassette consists of a GFP-tag linked to an IRES and a Neomycin/Kanamycin selection cassette. PCR fragments encoding the C-terminal or N-terminal GFP reporter cassette were generated using primers that each contain 50 nucleotide additional sequence homologous to the 5′ or 3′ sequence of the biomarker gene on the BAC (SEQ ID NOs: 42-51). These homologous sequences on both the 5′- and the 3′-ends of the PCR fragment allow Red/ET mediated site-specific recombination of the N-terminal or C-terminal GFP selection cassette at the 5′-end or 3′-end of the biomarker gene on the BAC. BAC strains that contain pRed/ET were grown at 37° C. for 45 min in the presence of L-arabinose to induce expression of the recombination enzymes. Subsequently, BAC strains were transformed with the GFP selection cassette PCR fragment by electroporation, incubated at 37° C. for 2 h to allow recombination of the PCR fragment with the BAC, and plated on kanamycin selection plates. Individual clones were analyzed for proper integration of the GFP cassette by PCR. Modified BACs were isolated using the Nucleobond PC100 DNA isolation kit (Macherey Nagel).

Creation of hiPSC Reporter Cell Lines

For hiPSC reporter cell lines, donor constructs were created from the BAC constructs containing the GFP selection cassette and homology arms matching the target sites within the reporter genes. PCR fragments were created using primers represented by SEQ ID NOs: 52-59. Furthermore, constructs containing Cas9 as well as a gRNA cutting near the START op STOP codon of the biomarker gene were created for gene targeting, as described in (Hsu et al., 2013), using the primers as represented by SEQ ID NOs:60-67.

hiPSC cells were seeded on Matrigel coated dishes 24 h prior to transfection. Donor constructs and gRNAs were transfected into hiPSC using lipofectamine 3000. Monoclonal hiPSC lines were selecting using neomycin and screened for integration of the construct by PCR.

Flow Cytometry

GPF reporter expression was generally determined by flow cytometry (Guava easyCyte 6HT, EMD Millipore). For this, differentiated cells were harvested as a single cell suspension using Tryple Select (Thermo) and resuspended in medium. Cell harvest was immediately followed by flow cytometry analysis.

Results

hiPSC were genetically modified to express OCT4-GFP from the endogenous locus and GFP expression was assed using flow cytometry. The cells were then differentiated as described above. At Day 0, OCT4 was highly expressed as expected in pluripotent cells (see FIG. 4A). During differentiation towards cardiomyocytes, both the average GFP intensity and the number of GFP positive cells reduced in the OCT4-GFP expressing clone (FIG. 4B). The OCT4-GFP cells were able to form beating cardiomyocytes, indicating that the gene targeting did not affect the differentiation potential of the hiPSC (results not shown).

### Example 4: Mouse ES Differentiation

Cardiomyocyte

For cardiomyocyte differentiation embryonic bodies were used, formed by hanging drops containing 750 cells in Iscoves's Modified Dulbecco's Medium (IMDM). Prior to hanging drop formation, cells were cultured on gelatine-coated dishes in IMDM supplemented with 20% FBS. Cells were detached with cell dissociation buffer (Gibco). After 3 days, embryonic bodies were transferred to a bacterial plate in differentiation medium (IMDM, 10% serum (unless specified otherwise), PS, NEAA and 2-mercapto ethanol). On day 5, the bodies were transferred to a 48-well plate in differentiation medium containing 2% serum, BMP, Activin A, Chir99021 and Xav939. On day 7 embryonic bodies were exposed to only Xav939 in differentiation medium and on day 11 all growth factors were removed and replaced with differentiation medium. Day 13 was used as endpoint for cardiomyocyte differentiation. On this day, beating bodies were quantified and RNA samples collected.

The results can be seen in FIG. 6A: OCT4, a marker of pluripotency was expressed at day 0 and, as expected, the expression decreased during differentiation. It was further found that BMP4 was a good marker for the middle stage of differentiation as the expression peaked around day 7, while Vegfr1 marked maturing cardiomyocytes and expression peaked around day 12. Exposure to the teratogenic agents 5′fluoracil, retinoic acid and diphenylhydantoin resulted in a decrease in beating cardiomyocytes after 13 days of differentiation, as can be seen in FIG. 5B.

Hepatocyte

Hepatocyte differentiation was started from a monolayer of mES cells on day −1. On day 0, differentiation medium supplemented with Activin A was added. On day 4, the medium was replaced with liver differentiation medium (DMEM containing 10% serum, PS, NEAA and 2-mercaptoethanol) with aFGF and sodium butyrate. On day 9, the medium was replaced with liver differentiation medium containing HGF. On day 14, cells were exposed to liver differentiation medium containing Dexamethasone and Oncostatin M. From day 17 onwards, liver differentiation medium was refreshed every 3-4 days. Endpoint of differentiation was set on day 21.

Proper differentiation of the mES cells was confirmed by a decrease in expression of the pluripotency marker gene OCT4, and expression of the FOXA2 and SOX17 genes during the intermediate stage of differentiation and expression peaks around day 10 (FIG. 6B).

### Example 5: mES Reporter Cell Lines

Creation of mES Reporter Cell Lines

mES cells were seeded on gelatin-coated culture dishes 24 h prior to transfection. Modified BACs were transfected into the mES cells using Lipofectamine 2000 (Invitrogen) as described previously (Poser et al., 2008, supra). Monoclonal mES cell lines were selected based on the level of induction of the GFP reporter after differentiation. GFP expression in differentiated cells was determined by flow cytometry.

Results

GFP-Ck18 mES reporter cells were created as described above; Ck18 is a known marker for mature hepatocytes. The reporter cells were differentiated towards hepatocytes as described above. During differentiation, the expression of GFP-Ck18 increased overtime (FIG. 7) indicating that the reporter cells behave like unmodified mES cells. To test the effect of teratogenic compounds on GFP-CK18 mES reporter cell line differentiation, the cells were exposed to the teratogenic compounds 5′Fluoracil and retinoic acid. After 21 days, fluorescence intensity of the hepatocyte specific CK-18-GFP reporter was measured and quantified (FIG. 8). GFP-Ck18 intensity was significantly reduced upon exposure to 5′Fluoracil and retinoic acid, indicating that the reporter cell line is a reliable way to measure teratogenic effects.

### REFERENCES

Chen, Y. F., Tseng, C. Y., Wang, H. W., Kuo, H. C., Yang, V. W., & Lee, O. K. (2012). Rapid generation of mature hepatocyte-like cells from human induced pluripotent stem cells by an efficient three-step protocol. Hepatology.
Den Hartogh, S. C., Schreurs, C., Monshouwer-Kloots, J. J., Davis, R. P., Elliott, D. A., Mummery, C. L., & Passier, R. (2015). Dual Reporter MESP1 mCherry/w-NKX2-5 eGFP/w hESCs Enable Studying Early Human Cardiac Differentiation. STEM CELLS.
Ellis-hutchings, R. G. & Ã, E. W. C. Whole Embryo Culture: A “New” Technique That Enabled Decades of Mechanistic Discoveries. 312, 304-312 (2010).
Hannan, N. R., Segeritz, C. P., Touboul, T., & Vallier, L. (2013). Production of hepatocyte-like cells from human pluripotent stem cells. Nature protocols, 8(2), 430.
Hendriks, G., Derr, R. S., Misovic, B., Morolli, B., Calleja, F. M. G. R., & Vrieling, H. (2016). The Extended ToxTracker Assay Discriminates Between Induction of DNA Damage, Oxidative Stress, and Protein Misfolding. Toxicological Sciences, 150(1), 190-203.
Hsu, P. D., Scott, D. A., Weinstein, J. A., Ran, F. A., Konermann, S., Agarwala, V., Zhang, F. (2013). DNA targeting specificity of RNA-guided Cas9 nucleases. Nature Biotechnology.
Lippmann, E. S., Estevez-Silva, M. C., & Ashton, R. S. (2014). Defined human pluripotent stem cell culture enables highly efficient neuroepithelium derivation without small molecule inhibitors. Stem Cells, 32(4), 1032-1042.
Piersma A. Session 3B: Teratological/Toxicological Aspects—Validation Studies, Industrial Applications. 7, 763-768 (1993).
Poser, I., Sarov, M., Hutchins, J. R. A., Hériché, J. K., Toyoda, Y., Pozniakovsky, A., Hyman, A. A. (2008). BAC TransgeneOmics: A high-throughput method for exploration of protein function in mammals. Nature Methods, 5(5), 409-415.
Uibel, F., Mühleisen, A., Köhle, C., Weimer, M., Stummann, T. C., Bremer, S., & Schwarz, M. (2010). ReProGlo: a new stem cell-based reporter assay aimed to predict embryotoxic potential of drugs and chemicals. Reproductive Toxicology, 30(1), 103-112.

1. An in vitro method of determining an effect of an agent on mammalian embryonic development, the method comprising:
a) providing at least seven types of reporter cells, wherein each type of reporter cell comprises a reporter sequence operatively linked to a different regulatory element of a gene;
b) contacting the at least seven type of reporter cells with the agent;
c) comparing the expression of the reporter sequences in the at least seven types of reporter cells contacted with the agent to a corresponding cell not contacted with the agent; and
d) determining that the agent has an effect on mammalian embryonic development if in step c) a difference in expression of the reporter sequences is detected between the reporter cells contacted with the agent and the corresponding cells not contacted with the agent for at least one type of reporter cell; and,
wherein the different regulatory elements for the at least seven types of reporter cells comprise:
a regulatory element of the OCT4 gene comprising a polynucleotide sequence that has at least 60% sequence identity with at least one of SEQ ID NO: 1 and SEQ ID NO: 21;
a regulatory element of the BMP4 gene, comprising a polynucleotide sequence that has at least 60% sequence identity at least one of SEQ ID NO: 2 and SEQ ID NO: 22;
a regulatory element of the MYH6 gene, comprising a polynucleotide sequence that has at least 60% sequence identity with at least one of SEQ ID NO: 3 and SEQ ID NO:23;
a regulatory element of the PAX6 gene, comprising a polynucleotide sequence that has at least 60% sequence identity with at least one of SEQ ID NO: 4 and SEQ ID NO: 24;
a regulatory element of the FOXA2 gene, comprising a polynucleotide sequence that has at least 60% sequence identity with at least one of SEQ ID NO: 5 and SEQ ID NO: 25;
a regulatory element of the AFP gene, comprising a polynucleotide sequence that has at least 60% sequence identity with at least one of SEQ ID NO: 8 and SEQ ID NO: 28; or
a regulatory element of the SOX1 gene, comprising a polynucleotide sequence that has at least 60% sequence identity with at least one of SEQ ID NO: 101 or SEQ ID NO: 103.

2. The method according to claim 1, wherein the method comprises contacting the agent in step b) with at least one additional type of reporter cells selected from the group consisting of:
a regulatory element of the SOX17 gene, comprising a polynucleotide sequence that has at least 60% sequence identity with at least one of SEQ ID NO: 6 and SEQ ID NO: 26;
a regulatory element of the ALB gene, comprising a polynucleotide sequence that has at least 60% sequence identity with at least one of SEQ ID NO: 7 and SEQ ID NO: 27;
a regulatory element of the Ck18 gene comprising a polynucleotide sequence that has at least 60% sequence identity with at least one of SEQ ID NO: 9 and SEQ ID NO: 29; and
a regulatory element of the Vegfr1 gene comprising a polynucleotide sequence that has at least 60% sequence identity with at least one of SEQ ID NO: 10 and SEQ ID NO: 30.

3. The method according to claim 1, wherein the reporter sequence is a gene encoding a protein.

4. The method according to claim 1, wherein the agent is a pharmaceutical agent, a chemical agent, a dye, an agrochemical agent, a cosmetic, a plasticizer or a food-ingredient.

5. The method according to claim 1, wherein the agent is a polypeptide, a peptide, a nucleic acid, a small molecule, or a natural product.

6. A kit of parts comprising:
at least seven types of reporter cell, wherein each type of reporter cell comprises a reporter sequence operatively linked to a regulatory element of a gene and wherein the at least seven type of reporter cells each comprise:
a regulatory element of the OCT4 gene comprising a polynucleotide sequence that has at least 60% sequence identity with at least one of SEQ ID NO: 1 and SEQ ID NO: 21;
a regulatory element of the BMP4 gene, comprising a polynucleotide sequence that has at least 60% sequence identity at least one of SEQ ID NO: 2 and SEQ ID NO: 22;
a regulatory element of the MYH6 gene, comprising a polynucleotide sequence that has at least 60% sequence identity with at least one of SEQ ID NO: 3 and SEQ ID NO:23;
a regulatory element of the PAX6 gene, comprising a polynucleotide sequence that has at least 60% sequence identity with at least one of SEQ ID NO: 4 and SEQ ID NO: 24;
a regulatory element of the FOXA2 gene, comprising a polynucleotide sequence that has at least 60% sequence identity with at least one of SEQ ID NO: 5 and SEQ ID NO: 25; or
a regulatory element of the AFP gene, comprising a polynucleotide sequence that has at least 60% sequence identity with at least one of SEQ ID NO: 8 and SEQ ID NO: 28.
a regulatory element of the SOX1 gene, comprising a polynucleotide sequence that has at least 60% sequence identity with at least one of SEQ ID NO: 101 or SEQ ID NO: 103.

7. The kit of parts according to claim 6, wherein the kit further comprises at least one additional type of reporter cell selected from the group consisting of:
a regulatory element of the SOX17 gene, comprising a polynucleotide sequence that has at least 60% sequence identity with at least one of SEQ ID NO: 6 and SEQ ID NO: 26;
a regulatory element of the ALB gene, comprising a polynucleotide sequence that has at least 60% sequence identity with at least one of SEQ ID NO: 7 and SEQ ID NO: 27;
a regulatory element of the Ck18 gene comprising a polynucleotide sequence that has at least 60% sequence identity with at least one of SEQ ID NO: 9 and SEQ ID NO: 29; and
a regulatory element of the Vegfr1 gene comprising a polynucleotide sequence that has at least 60% sequence identity with at least one of SEQ ID NO: 10 and SEQ ID NO: 30.

8. A combination of at least seven transgenic non-human mammals, comprising at least one cell comprising a reporter sequence operatively linked to a different regulatory element and wherein the regulatory element is selected from:
a regulatory element of the OCT4 gene comprising a polynucleotide sequence that has at least 60% sequence identity with at least one of SEQ ID NO: 1 and SEQ ID NO: 21;
a regulatory element of the BMP4 gene, comprising a polynucleotide sequence that has at least 60% sequence identity at least one of SEQ ID NO: 2 and SEQ ID NO: 22;
a regulatory element of the MYH6 gene, comprising a polynucleotide sequence that has at least 60% sequence identity with at least one of SEQ ID NO: 3 and SEQ ID NO: 23;
a regulatory element of the PAX6 gene, comprising a polynucleotide sequence that has at least 60% sequence identity with at least one of SEQ ID NO: 4 and SEQ ID NO: 24;
a regulatory element of the FOXA2 gene, comprising a polynucleotide sequence that has at least 60% sequence identity with at least one of SEQ ID NO: 5 and SEQ ID NO: 25;
a regulatory element of the AFP gene, comprising a polynucleotide sequence that has at least 60% sequence identity with at least one of SEQ ID NO: 8 and SEQ ID NO: 28; and
a regulatory element of the SOX1 gene, comprising a polynucleotide sequence that has at least 60% sequence identity with at least one of SEQ ID NO: 101 or SEQ ID NO: 103.

9. The combination according to claim 8, wherein the combination comprises one or more additional transgenic non-human mammals selected from the group consisting of:
a transgenic non-human mammal comprising a reporter sequence operatively linked to a different regulatory element and wherein the regulatory element is a regulatory element of the SOX17 gene, comprising a polynucleotide sequence that has at least 60% sequence identity with at least one of SEQ ID NO: 6 and SEQ ID NO: 26;
a transgenic non-human mammal comprising a reporter sequence operatively linked to a different regulatory element and wherein the regulatory element is a regulatory element of the ALB gene, comprising a polynucleotide sequence that has at least 60% sequence identity with at least one of SEQ ID NO: 7 and SEQ ID NO: 27;
a transgenic non-human mammal comprising a reporter sequence operatively linked to a different regulatory element and wherein the regulatory element is a regulatory element of the Ck18 gene comprising a polynucleotide sequence that has at least 60% sequence identity with at least one of SEQ ID NO: 9 and SEQ ID NO: 29; and
a transgenic non-human mammal comprising a reporter sequence operatively linked to a different regulatory element and wherein the regulatory element is a regulatory element of the Vegfr1 gene comprising a polynucleotide sequence that has at least 60% sequence identity with at least one of SEQ ID NO: 10 and SEQ ID NO: 30.

10.-11. (canceled)

12. The method according to claim 3, wherein the reporter sequence encodes a protein that is readily detectable.

13. The combination according to claim 8, wherein the transgenic non-human mammals are rodents.
